(19) United States
(12) Patent Application Publication
(10) Pub. No.: US 2014/0099709 A1
(43) Pub. Date: Apr. 10, 2014

(54) ENGINEERED THREE-DIMENSIONAL CONNECTIVE TISSUE CONSTRUCTS AND METHODS OF MAKING THE SAME

(71) Applicant: ORGANOVO, INC., San Diego, CA (US)

(72) Inventors: Sharon C. Presnell, San Diego, CA (US); Benjamin R. Shepherd, San Diego, CA (US); Albert J. Evinger, III, Pomona, CA (US)

(73) Assignee: ORGANOVO, INC., San Diego, CA (US)

(21) Appl. No.: 13/801,780

(22) Filed: Mar. 13, 2013

Related U.S. Application Data
(60) Provisional application No. 61/661,768, filed on Jun. 19, 2012.

(51) Int. Cl. C12N 5/077

(52) U.S. Cl. CPC C12N 5/0654
USPC 435/347; 435/325; 435/377; 435/373

(57) ABSTRACT

Disclosed are engineered, living, three-dimensional connective tissue constructs comprising connective tissue cells. In some embodiments, the connective tissue cells are derived from multi-potent cells such as mesenchymal stem/stromal cells. In some embodiments, the cells are cohered to one another. In some embodiments, the multi-potent cells have been exposed to one or more differentiation signals to provide a living, three-dimensional connective tissue construct. In some embodiments, the constructs are substantially free of pre-formed scaffold at the time of use. Also disclosed are implants for engraftment, arrays of connective tissue constructs for in vitro experimentation, as well as methods of making the same.

### CROSS-REFERENCE TO RELATED APPLICATIONS

This application claims the benefit of U.S. application Ser. No. 61/661,768, filed Jun. 19, 2012, which is hereby incorporated by reference in its entirety.

### BACKGROUND OF THE INVENTION

A number of pressing problems confront the healthcare industry. As of June 2012 there were 114,636 patients registered by United Network for Organ Sharing (UNOS) as needing an organ transplant. According to UNOS, between January and March 2012 only 6,838 transplants were performed. Each year more patients are added to the UNOS list than transplants are performed, resulting in a net increase in the number of patients waiting for a transplant.

Additionally, the research and development cost of a new pharmaceutical compound is approximately $1.8 billion. See Paul, et al. (2010). How to improve R&D productivity: the pharmaceutical industry's grand challenge. Nature Reviews Drug Discovery 9(3):203-214. Drug discovery is the process by which drugs are discovered and/or designed. The process of drug discovery generally involves at least the steps of: identification of candidates, synthesis, characterization, screening, and assays for therapeutic efficacy. Despite advances in technology and understanding of biological systems, drug discovery is still a lengthy, expensive, and inefficient process with low rate of new therapeutic discovery.

### SUMMARY OF THE INVENTION

In one aspect, disclosed herein are engineered, living, three-dimensional connective tissue constructs comprising: connective tissue cells cohered to one another to provide a living, three-dimensional connective tissue construct; wherein the construct is substantially free of pre-formed scaffold. In some embodiments, the construct is substantially free of any pre-formed scaffold at the time of use. In some embodiments, the construct is non-innervated. In some embodiments, the connective tissue cells comprise connective tissue cells derived in vitro from multi-potent cells. In some embodiments, the multi-potent cells comprise one or more of: tissue-specific progenitors, mesenchymal stem/stromal cells, induced pluripotent stem cells, and embryonic stem cells. In some embodiments, the multi-potent cells are derived from mammalian adipose tissue. In other embodiments, the multi-potent cells are derived from mammalian bone marrow. In yet other embodiments, the multi-potent cells are derived from a non-adipose, non-bone marrow tissue source. In some embodiments, the multi-potent cells were exposed to one or more differentiation signals before fabrication of the construct. In some embodiments, the multi-potent cells were exposed to one or more differentiation signals during fabrication of the construct. In some embodiments, the multi-potent cells were exposed to one or more differentiation signals after fabrication of the construct. In some embodiments, the construct was bioprinted. In further embodiments, the construct further comprises an extrusion compound, the extrusion compound improving the suitability of the cells for bioprinting. In some embodiments, the connective tissue is selected from the group consisting of: bone, cartilage, tendon, and ligament. In some embodiments, the construct further comprises one or more of the following cell types: vascular, endothelial, fibroblasts, pericytes, stem/progenitor cells, immune cells. In some embodiments, the construct is substantially in the form of a sheet, patch, ring, tube, cube, polyhedron, or sphere. In some embodiments, the construct is substantially in the form of a shape that mimics the shape or architecture of a native human connective tissue in vivo. In some embodiments, the construct is for implantation in a subject at a site of injury, disease, or degeneration. In some embodiments, the construct further comprises one or more of discrete filler bodies, each filler body comprising a biocompatible material, wherein the one or more filler body creates a gap or space in the cohered cells. In further embodiments, each filler body substantially resists migration and ingrowth of cells.

In another aspect, disclosed herein are arrays of engineered, living, three-dimensional connective tissue constructs, each construct fabricated by a process comprising: exposing multi-potent cells to one or more differentiation signals to provide a living, three-dimensional connective tissue construct; wherein each connective tissue construct is substantially free of pre-formed scaffold; wherein each connective tissue construct is maintained in culture. In some embodiments, each construct is substantially free of any pre-formed scaffold at the time of use. In some embodiments, each construct is non-innervated. In some embodiments, the multi-potent cells comprise one or more of: tissue-specific progenitors, mesenchymal stem/stromal cells, induced pluripotent stem cells, and embryonic stem cells. In some embodiments, the multi-potent cells are derived from mammalian adipose tissue. In other embodiments, the multi-potent cells are derived from mammalian bone marrow. In yet other embodiments, the multi-potent cells are derived from a non-adipose, non-bone marrow tissue source. In some embodiments, the multi-potent cells were exposed to the one or more differentiation signals before fabrication of the construct. In some embodiments, the multi-potent cells were exposed to the one or more differentiation signals during fabrication of the construct. In some embodiments, the multi-potent cells were exposed to the one or more differentiation signals after fabrication of the construct. In some embodiments, each construct was bioprinted. In some embodiments, the connective tissue is selected from the group consisting of: bone, cartilage, tendon, and ligament. In some embodiments, one or more connective tissue constructs further comprises one or more of the following cell types: endothelial cells, fibroblasts, stem/progenitor cells, pericytes, satellite cells, or vascular cells. In some embodiments, one or more connective tissue constructs are compound tissue constructs comprising one or more connective tissues. In further embodiments, one or more connective tissue constructs are compound tissue constructs comprising connective tissue and a non-connective tissue. In still further embodiments, one or more connective tissue constructs are compound tissue constructs comprising bone tissue and a non-connective tissue. In some embodiments, the arrays are for use in in vitro assays. In further embodiments, the arrays are for use in one or more of: drug discovery, drug testing, toxicology testing, disease modeling, three-dimensional biology studies, and cell screening. In some embodiments, the one or more differentiation signals comprise mechanical, biomechanical, soluble, or physical signals, or combinations thereof. In some embodiments, one or more constructs further comprises one or more discrete filler bodies, each filler body comprising a biocompatible material, wherein the one or more filler body creates a gap or space in the cohered cells. In further embodiments, each filler body substantially resists migration and ingrowth of cells.

In another aspect, disclosed herein are methods of fabricating a living, three-dimensional connective tissue construct comprising: incubating a bio-ink, comprising multi-potent cells that have been deposited on a support and exposed to one or more differentiation signals, to allow the bio-ink to cohere and to form a living, three-dimensional connective tissue construct, wherein said incubation has a duration of about 1 hour to about 30 days. In some embodiments, the multi-potent cells comprise one or more of: mesenchymal stem/stromal cells, induced pluripotent stem cells, and embryonic stem cells. In some embodiments, the multi-potent cells are derived from mammalian adipose tissue. In other embodiments, the multi-potent cells are derived from mammalian bone marrow. In yet other embodiments, the multi-potent cells are derived from a non-adipose, non-bone marrow tissue source. In some embodiments, the connective tissue cells are exposed to one or more differentiation signals at one or more time intervals between about 1-21 days before depositing the bio-ink onto the support to about 1-21 days after depositing the bio-ink onto the support. In some embodiments, the bio-ink is deposited by bioprinting. In some embodiments, the construct is substantially free of any pre-formed scaffold at the time of use. In some embodiments, the construct is non-innervated. In some embodiments, the connective tissue is selected from the group consisting of: bone, cartilage, tendon, and ligament. In some embodiments, the bio-ink further comprises one or more of the following cell types: vascular, endothelial, fibroblasts, pericytes, stem/progenitor cells, immune cells. In some embodiments, the bio-ink further comprises an extrusion compound. In some embodiments, the one or more differentiation signals comprise mechanical, biomechanical, soluble, or physical signals, or combinations thereof. In some embodiments, the method further comprises the step of depositing one or more discrete filler bodies, each filler body comprising a biocompatible material, wherein the one or more filler body creates a gap or space in the cohered cells. In further embodiments, each filler body substantially resists migration and ingrowth of cells. In some embodiments, the method further comprises the step of assembling a plurality of living, three-dimensional connective tissue constructs into an array by spatially confining the constructs onto or within a biocompatible surface. In some embodiments, the construct is suitable for implantation in a subject at a site of injury, disease, or degeneration.

In another aspect, disclosed herein are methods of fabricating a living, three-dimensional connective tissue construct comprising the steps of: preparing bio-ink comprising multi-potent cells; depositing the bio-ink onto a support; and incubating the bio-ink to allow the bio-ink to cohere and to form a living, three-dimensional connective tissue construct, wherein said incubation has a duration of about 1 hour to about 30 days; with the proviso that the multi-potent cells are exposed to one or more differentiation signals. In some embodiments, the multi-potent cells comprise one or more of: mesenchymal stem/stromal cells, induced pluripotent stem cells, and embryonic stem cells. In some embodiments, the multi-potent cells are derived from mammalian adipose tissue. In other embodiments, the multi-potent cells are derived from mammalian bone marrow. In yet other embodiments, the multi-potent cells are derived from a non-adipose, non-bone marrow tissue source. In some embodiments, the connective tissue cells are exposed to one or more differentiation signals at one or more time intervals between about 1-21 days before depositing the bio-ink onto the support to about 1-21 days after depositing the bio-ink onto the support. In some embodiments, the bio-ink is deposited by bioprinting. In some embodiments, the construct is substantially free of any pre-formed scaffold at the time of use. In some embodiments, the construct is non-innervated. In some embodiments, the connective tissue is selected from the group consisting of: bone, cartilage, tendon, and ligament. In some embodiments, the bio-ink further comprises one or more of the following cell types: vascular, endothelial, fibroblasts, pericytes, stem/progenitor cells, immune cells. In some embodiments, the bio-ink further comprises an extrusion compound. In some embodiments, the one or more differentiation signals comprise mechanical, biomechanical, soluble, or physical signals, or combinations thereof. In some embodiments, the method further comprises the step of depositing one or more discrete filler bodies, each filler body comprising a biocompatible material, wherein the one or more filler body creates a gap or space in the cohered cells. In further embodiments, each filler body substantially resists migration and ingrowth of cells. In some embodiments, the method further comprises the step of assembling a plurality of living, three-dimensional connective tissue constructs into an array by spatially confining the constructs onto or within a biocompatible surface. In some embodiments, the construct is suitable for implantation in a subject at a site of injury, disease, or degeneration.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a non-limiting exemplary timeline of stem cell differentiation; in this case, a timeline of differentiation demonstrating pre-differentiation, peri-differentiation, and post-differentiation periods wherein stem cells are incubated in contact with osteogenic differentiation media.

FIG. 2A is an image depicting a non-limiting example of bioprinted MSC constructs; in this case, in situ alkaline phosphatase staining of bioprinted MSC constructs cultured in differentiation media. This figure demonstrates expression of alkaline phosphatase in constructs exposed to differentiation media.

FIG. 2B is an image depicting a non-limiting example of bioprinted MSC constructs; in this case, in situ alkaline phosphatase staining of bioprinted MSC constructs cultured in basal MSC culture media. No expression of alkaline phosphatase was observed in constructs exposed to basal MSC culture media.

FIG. 2C is a photomicrograph at 20× depicting a non-limiting example of bioprinted MSC constructs; in this case, bioprinted MSC constructs cultured in differentiation media immediately post-printing and stained with Alizarin Red S to identify calcium deposits.

FIG. 2D is a photomicrograph at 20× depicting a non-limiting example of bioprinted MSC constructs; in this case, bioprinted MSC constructs cultured in basal MSC culture media immediately post-printing and stained with Alizarin Red S. No calcium deposits were observed in constructs exposed to basal MSC culture media.

FIG. 3 is a non-limiting photomicrograph of immunofluorescence staining of tissue sections of formalin-fixed paraffin-embedded MSC constructs after 5 d of post-bioprint incubation in differentiation media detecting the expression of osteopontin, indicative of MSC differentiation and osteogenesis.

FIGS. 4A and 4B are photomicrographs at 20× depicting mesenchymal stem cell-containing constructs that were bioprinted and cultured in either osteogenic differentiation medium or only basal mesenchymal stem cell culture media. Histological alkaline phosphatase staining of bioprinted constructs was utilized to detect osteoblast activity. FIG. 4A illustrates little or no expression of alkaline phosphatase in constructs exposed only to basal mesenchymal stem cell culture media. Whereas FIG. 4B illustrates expression of alkaline phosphatase in constructs exposed to osteogenic differentiation medium.

### DETAILED DESCRIPTION OF THE INVENTION

At the beginning of 2008, 75,834 people were registered as needing a kidney; at the end of that year, the number had grown to 80,972. 16,546 kidney transplants were performed that year, but 33,005 new patients were added to the list. The 2008 transplant rate for a patient registered by UNOS as needing a kidney was 20%. The mortality rate of waitlist patients was 7%. Furthermore, many individuals suffer from chronic degenerative diseases for which transplantation is not a current healthcare paradigm. Thus, living, functional connective tissues (bone, tendon, ligament, etc.) would be of great clinical value. There is a need for materials, tools, and techniques that facilitate application of regenerative medicine and tissue engineering technologies to relieving the urgent need for implantable tissues and organs. More specifically, there is a need for implantable tissues and organs that are suitable for wound repair, tissue repair, tissue augmentation, organ repair, and organ replacement. Just as important, there is a need for materials, tools, and techniques that substantially increase the number and quality of innovative, cost-effective new medicines, without incurring unsustainable research and development costs.

Previous models have been focused on providing engineered tissue constructs by seeding cells onto a three-dimensional scaffold material that is pre-formed and shaped to accommodate the intended application. Cells seeded onto scaffold materials have been primary cells, cell lines, engineered cells, and/or stem/progenitor cells. When multipotential stem or progenitor cells are utilized, they have either undergone a differentiation program in two-dimensional monolayer culture prior to seeding on a three-dimensional scaffold material, or they have first been seeded onto a scaffold material and then been subjected to a differentiation program, in situ or in vitro, to generate the desired tissue. The traditional approach is both laborious and inefficient in terms of cell yield, the time required for terminal differentiation of the cells within the construct, and the overall cellularity of the resulting three-dimensional structure.

The invention relates to the field of regenerative medicine and tissue engineering. More particularly, the invention relates to living, three-dimensional connective tissue constructs, arrays thereof, and methods of fabrication. The connective tissue constructs are useful as implantable/therapeutic devices or as arrayed tissue constructs for in vitro experimentation (i.e., drug development, compound screening, toxicology and disease modeling).

Disclosed herein, in certain embodiments, are engineered, living, three-dimensional connective tissue constructs comprising: connective tissue cells cohered to one another to provide a living, three-dimensional connective tissue construct; wherein the construct is substantially free of pre-formed scaffold.

Also disclosed herein, in certain embodiments, are arrays of engineered, living, three-dimensional connective tissue constructs, each construct fabricated by a process comprising: exposing multi-potent cells to one or more differentiation signals to provide a living, three-dimensional connective tissue construct; wherein each connective tissue construct is substantially free of pre-formed scaffold; wherein each connective tissue construct is maintained in culture.

Also disclosed herein, in certain embodiments, are methods of fabricating a living, three-dimensional connective tissue construct comprising: incubating a bio-ink, comprising multi-potent cells that have been deposited on a support and exposed to one or more differentiation signals, to allow the bio-ink to cohere and to form a living, three-dimensional connective tissue construct, wherein said incubation has a duration of about 1 hour to about 30 days.

Also disclosed herein, in certain embodiments, are methods of fabricating a living, three-dimensional connective tissue construct comprising the steps of: preparing bio-ink comprising multi-potent cells; depositing the bio-ink onto a support; and incubating the bio-ink to allow the bio-ink to cohere and to form a living, three-dimensional connective tissue construct, wherein said incubation has a duration of about 1 hour to about 30 days; with the proviso that the multi-potent cells are exposed to one or more differentiation signals.

### Certain Definitions

Unless otherwise defined, all technical and scientific terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

As used herein, “array” means a scientific tool including an association of multiple elements spatially arranged to allow a plurality of tests to be performed on a sample, one or more tests to be performed on a plurality of samples, or both.

As used herein, “assay” means a procedure for testing or measuring the presence or activity of a substance (e.g., a chemical, molecule, biochemical, protein, hormone, or drug, etc.) in an organic or biological sample (e.g., cell aggregate, tissue, organ, organism, etc.).

As used herein, “biocompatible” means posing limited risk of injury or toxicity to cells. As presented in the specification and claims, “biocompatible multi-well containers” and “biocompatible membranes” pose limited risk of injury or toxicity to mammalian cells, but the definition does not extend to imply that these biocompatible elements could be implanted in vivo into a mammal.

As used herein, “bioprinting” means utilizing three-dimensional, precise deposition of cells (e.g., cell solutions, cell-containing gels, cell suspensions, cell concentrations, multicellular aggregates, multicellular bodies, etc.) via methodology that is compatible with an automated, computer-aided, three-dimensional prototyping device (e.g., a bioprinter).

As used herein, “cohere,” “cohered,” and “cohesion” refer to cell-cell adhesion properties that bind cells, multicellular aggregates, multicellular bodies, and layers thereof. The terms are used interchangeably with “fuse,” “fused,” and “fusion.”

As used herein, “multi-potent cells” refers to cells that are capable of undergoing differentiation to two or more cell types. Multi-potent cells include, for example, mesenchymal stem/stromal cells, induced pluripotent stem cells, and embryonic stem cells.

As used herein, “mesenchymal stem/stromal cells” refers to a specific type of multi-potent cells that potentially differentiate into a variety of cell types and exhibit the properties and characteristics described further herein. In some embodiments, the terms “mesenchymal stem cells” and “mesenchymal stromal cells” are used interchangeably with “mesenchymal stem/stromal cells.”

As used herein, “scaffold” refers to synthetic scaffolds such as polymer scaffolds and porous hydrogels, non-synthetic scaffolds such as pre-formed extracellular matrix layers and decellularized tissues, and any other type of pre-formed scaffold that is integral to the physical structure of the engineered tissue and/or organ and not able to be removed from the tissue and/or organ without damage/destruction of the tissue and/or organ. The term “scaffoldless,” therefore, is intended to imply that scaffold is not an integral part of the engineered tissue at the time of use, either having been removed or remaining as an inert component of the engineered tissue. “Scaffoldless” is used interchangeably with “scaffold-free” and “free of pre-formed scaffold.”

As used herein, “subject” means any individual, which may be a human, a non-human animal, any mammal, or any vertebrate. The term is interchangeable with “patient,” “recipient” and “donor.”

As used herein, “tissue” means an aggregate of cells. Examples of tissues include, but are not limited to, connective tissue (e.g., areolar connective tissue, dense connective tissue, elastic tissue, reticular connective tissue, and adipose tissue), muscle tissue (e.g., skeletal muscle, smooth muscle and cardiac muscle), genitourinary tissue, gastrointestinal tissue, pulmonary tissue, bone tissue, nervous tissue, and epithelial tissue (e.g., simple epithelium and stratified epithelium), ectodermal tissue, endodermal tissue, or mesodermal tissue.

### Tissue Engineering

Tissue engineering is an interdisciplinary field that applies and combines the principles of engineering and life sciences toward the development of biological substitutes that restore, maintain, or improve tissue function through augmentation, repair, or replacement of an organ. The basic approach to classical tissue engineering is to seed living cells into a biocompatible and eventually biodegradable environment (e.g., a scaffold), and then culture this construct in a bioreactor so that the initial cell population can expand further and mature to generate the target tissue upon implantation. With an appropriate scaffold that mimics the biological extracellular matrix (ECM), the developing tissue may adopt both the form and function of the desired organ after in vitro and in vivo maturation. However, achieving high enough cell density with a native tissue-like architecture is challenging due to the limited ability to control the distribution and spatial arrangement of the cells throughout the scaffold. These limitations may result in tissues or organs with poor mechanical properties and/or insufficient function. Additional challenges exist with regard to biodegradation of the scaffold, entrapment of residual polymer, and industrial scale-up of manufacturing processes. Scaffoldless approaches have been attempted. Current scaffoldless approaches are subject to several limitations:

Complex geometries, such as multi-layered structures wherein each layer comprises a different cell type, or comprises specific cellular compartments that are spatially confined, may require definitive, high-resolution placement of cell types within a specific architecture to reproducibly achieve a native tissue-like outcome.

Scale and geometry are limited by diffusion and/or the requirement for functional vascular networks for nutrient supply.

The viability of the tissues may be compromised by confinement material that limits diffusion and restricts the cells' access to nutrients.

Disclosed herein, in certain embodiments, are engineered tissues, engineered connective tissue constructs, arrays thereof, and methods of fabrication. The tissue engineering methods disclosed herein have the following advantages:

They are capable of producing cell-comprising tissues and/or organs with a broad array of complex, three-dimensional topologies.

They mimic the environmental conditions of the natural tissue-forming processes by exploiting principles of developmental biology.

They are compatible with automated means of manufacturing and are scalable.

Bioprinting enables improved methods of generating cell-comprising implantable tissues that are useful in tissue repair, tissue augmentation, and tissue replacement. Bioprinting further enables improved methods of generating micro-scale tissue analogs including those useful for in vitro assays.

### Bioprinting

In some embodiments, at least one component of the engineered tissues, including connective tissue constructs, and arrays thereof were bioprinted. In further embodiments, the engineered tissues were entirely bioprinted. In still further embodiments, bioprinted constructs are made with a method that utilizes a rapid prototyping technology based on three-dimensional, automated, computer-aided deposition of cells, including cell solutions, cell suspensions, cell-comprising gels or pastes, cell concentrations, multicellular bodies (e.g., cylinders, spheroids, ribbons, etc.) (collectively “bio-ink”), and, optionally, confinement material onto a biocompatible surface (e.g., composed of hydrogel and/or a porous membrane) by a three-dimensional delivery device (e.g., a bioprinter). As used herein, in some embodiments, the term “engineered,” when used to refer to tissues and/or organs means that cells, cell solutions, cell suspensions, cell-comprising gels or pastes, cell concentrates, multicellular aggregates (e.g., bio-ink), and layers thereof are positioned to form three-dimensional structures by a computer-aided device (e.g., a bioprinter) according to a computer script. In further embodiments, the computer script is, for example, one or more computer programs, computer applications, or computer modules. In still further embodiments, three-dimensional tissue structures form through the post-printing fusion of cells or bio-ink similar to self-assembly phenomena in early morphogenesis.

While a number of methods are available to arrange cells, bio-ink (e.g., multicellular bodies), and/or layers thereof on a biocompatible surface to produce a three-dimensional structure including manual placement, positioning by an automated, computer-aided instrument such as a bioprinter is advantageous. Advantages of delivery of cells or multicellular bodies with this technology include rapid, accurate, and reproducible placement of cells or bio-ink (e.g., multicellular bodies) to produce constructs exhibiting planned or pre-determined orientations or patterns of cells, bio-ink (e.g., multicellular bodies), and/or layers thereof with various compositions. Advantages also include assured high cell density, while minimizing cell damage.

In some embodiments, the method of bioprinting is continuous and/or substantially continuous. A non-limiting example of a continuous bioprinting method is to dispense bio-ink from a bioprinter via a dispense tip (e.g., a syringe, capillary tube, etc.) connected to a reservoir of bio-ink. In further non-limiting embodiments, a continuous bioprinting method is to dispense bio-ink in a repeating pattern of functional units. In various embodiments, a repeating functional unit has any suitable geometry, including, for example, circles, squares, rectangles, triangles, polygons, and irregular geometries. In further embodiments, a repeating pattern of bioprinted function units comprises a layer and a plurality of layers are bioprinted adjacently (e.g., stacked) to form an engineered tissue or organ. In various embodiments, 2, 3, 4, 5, 6, 7, 8, 9, 10, 11, 12, 13, 14, 15, or more layers are bioprinted adjacently (e.g., stacked) to form an engineered tissue or organ.

In some embodiments, a bioprinted functional unit repeats in a tessellated pattern. A “tessellated pattern” is a plane of figures that fills the plane with no overlaps and no gaps. An advantage of continuous and/or tessellated bioprinting can include an increased productivity of bioprinted tissue. Another non-limiting potential advantage can be eliminating the need to align the bioprinter with previously deposited elements of bio-ink. Continuous bioprinting may also facilitate printing larger tissues from a large reservoir of bio-ink, optionally using a syringe mechanism.

Methods in continuous bioprinting may involve optimizing and/or balancing parameters such as print height, pump speed, robot speed, or combinations thereof independently or relative to each other. In one example, the bioprinter head speed for deposition was 3 mm/s, with a dispense height of 0.5 mm for the first layer and dispense height was increased 0.4 mm for each subsequent layer. In some embodiments, the dispense height is approximately equal to the diameter of the bioprinter dispense tip. Without limitation a suitable and/or optimal dispense distance does not result in material flattening or adhering to the dispensing needle. In various embodiments, the bioprinter dispense tip has an inner diameter of about, 20, 50, 100, 150, 200, 250, 300, 350, 400, 450, 500, 550, 600, 650, 700, 750, 800, 850, 900, 950, 1000 μm, or more, including increments therein. In various embodiments, the bio-ink reservoir of the bioprinter has a volume of about 0.5, 1, 2, 3, 4, 5, 6, 7, 8, 9, 10, 15, 20, 25, 30, 35, 40, 45, 50, 55, 60, 65, 70, 75, 80, 85, 90, 95, 100 cubic centimeters, or more, including increments therein. The pump speed may be suitable and/or optimal when the residual pressure build-up in the system is low. Favorable pump speeds may depend on the ratio between the cross-sectional areas of the reservoir and dispense needle with larger ratios requiring lower pump speeds. In some embodiments, a suitable and/or optimal print speed enables the deposition of a uniform line without affecting the mechanical integrity of the material.

The inventions disclosed herein include business methods. In some embodiments, the speed and scalability of the techniques and methods disclosed herein are utilized to design, build, and operate industrial and/or commercial facilities for production of engineered tissues and/or organs for implantation or use in generation of cell-based tools for research and development, such as in vitro assays. In further embodiments, the engineered tissues and/or organs and arrays thereof are produced, stored, distributed, marketed, advertised, and sold as, for example, implantable tissues for wound repair, tissue repair, tissue augmentation, organ repair, and organ replacement. In still further embodiments, the engineered tissues and/or organs and arrays thereof are produced, stored, distributed, marketed, advertised, and sold as, for example, cellular arrays (e.g., microarrays or chips), tissue arrays (e.g., microarrays or chips), and kits for biological assays and high-throughput drug screening. In other embodiments, the engineered tissues and/or organs and arrays thereof are produced and utilized to conduct biological assays and/or drug screening as a service.

### Engineered Tissues Including Connective Tissue Constructs

Disclosed herein, in some embodiments, are living, three-dimensional tissue constructs comprising: connective tissue cells cohered to one another; wherein the construct is substantially free of pre-formed scaffold. In further embodiments the construct is substantially free of pre-formed scaffold at the time of fabrication and/or the time of use. In some embodiments, the tissues are connective tissue constructs. Therefore, also disclosed herein, in some embodiments, are living, three-dimensional connective tissue constructs comprising: connective tissue cells cohered to one another to provide a living, three-dimensional connective tissue construct; wherein the construct is substantially free of pre-formed scaffold at the time of use. In some embodiments, the connective tissue cells are derived from multi-potent cells such as mesenchymal stem/stromal cells, induced pluripotent stem cells, and/or embryonic stem cells.

In some embodiments, the engineered tissues, including connective tissues, are bioprinted, a methodology described herein. In further embodiments, the tissues are substantially free of any pre-formed scaffold as described further herein at the time of printing and/or the time of use. In some embodiments, as a result of being fabricated by tissue engineering techniques, including bioprinting, the tissues of the present invention are further distinguished from tissues developed in vivo, as part of an organism. In some embodiments, the engineered tissues described herein are characterized by structural and architectural differences from tissues developed in vivo, as part of an organism. By way of non-limiting example, in some embodiments, the engineered tissues described herein are non-innervated or lack a functional nervous system. By way of further non-limiting example, in some embodiments, the engineered tissues described herein lack a functional immune system. By way of further non-limiting example, in some embodiments, the engineered tissues described herein lack blood components.

In some embodiments, the engineered tissues, including connective tissues, include any type of mammalian cell. In various further embodiments, the tissues, including connective tissues, include 1, 2, 3, 4, 5, 6, 7, 8, 9, 10, 11, 12, 13, 14, 15, 16, 17, 18, 19, 20 or more cell types. In some embodiments, the tissues include stem cells. In further embodiments, the tissues include multi-potent cells such as mesenchymal stem/stromal cells, induced pluripotent stem cells, and/or embryonic stem cells.

In some embodiments, some or all of the multi-potent cells (e.g., mesenchymal stem/stromal cells, induced pluripotent stem cells, embryonic stem cells, etc.) are undifferentiated and multi-potent at the time of fabrication of the tissue. In further embodiments, some or all of the multi-potent cells are partially differentiated, to some degree, toward one or more tissue-specific phenotypes consistent with, for example, osteocytes, chondrocytes, or adipose cells at the time of fabrication of the tissue. In further embodiments, some or all of the multi-potent cells are completely differentiated to one or more tissue-specific phenotypes consistent with, for example, osteocytes, chondrocytes, or adipose cells at the time of fabrication of the tissue.

In some embodiments, the multi-potent cells (e.g., mesenchymal stem/stromal cells, induced pluripotent stem cells, embryonic stem cells, etc.) have been exposed to one or more differentiation signals to provide a living, three-dimensional connective tissue construct. In various embodiments, the multi-potent cells have been exposed to one or more differentiation signals, at one or more time intervals before, during, or after depositing the bio-ink to form a tissue construct. In further embodiments, the multi-potent cells have been exposed to one or more differentiation signals before preparation of bio-ink using the cells. In further embodiments, the multi-potent cells have been exposed to one or more differentiation signals before fabrication of tissue using the bio-ink. In further embodiments, the multi-potent cells have been exposed to one or more differentiation signals after fabrication of tissue using the bio-ink.

In other embodiments, the tissues further include, for example, mammalian endothelial cells and/or mammalian fibroblasts. In some embodiments, the cells of the engineered tissues, including connective tissues, are “cohered” or “adhered” to one another. In further embodiments, cohesion or adhesion refers to cell-cell adhesion properties that bind cells and bio-ink (e.g., multicellular aggregates, multicellular bodies, etc.), and/or layers thereof.

The engineered tissues, including connective tissue constructs, in various embodiments, are any suitable size. In some embodiments, the size of bioprinted tissues, including connective tissue constructs, change over time. In further embodiments, a bioprinted tissue shrinks or contracts after bioprinting due to, for example, cell migration, cell death, intercellular interactions, contraction, or other forms of shrinkage. In other embodiments, a bioprinted tissue grows or expands after bioprinting due to, for example, cell migration, cell growth and proliferation, cell maturation, or other forms of expansion.

In some embodiments, the physical dimensions of the engineered tissues, including connective tissue constructs, are limited by the capacity for nutrients, including oxygen, to diffuse into the interior of the construct. In various embodiments, the engineered tissues, including connective tissue constructs, are at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950, or 1000 μm, including increments therein, in their smallest dimension at the time of bioprinting. In various embodiments, the engineered tissues, including connective tissue constructs, are at least about 0.25, 0.5, 0.75, 1.0, 1.25, 1.5, 1.75, 2.0, 2.25, 2.5, 2.75, 3.0, 3.25, 3.5, 3.75, 4.0, 4.25, 4.5, 4.75, or 5.0 mm, including increments therein, in their smallest dimension at the time of bioprinting. In further embodiments, the engineered tissues, including connective tissue constructs, are between about 50 μm and about 500 μm in their smallest dimension at the time of bioprinting.

In some embodiments, the physical dimensions of the engineered tissues, including connective tissue constructs, are about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 mm, including increments therein, wide.

In some embodiments, the physical dimensions of the engineered tissues, including connective tissue constructs, are about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 mm, including increments therein, long.

The engineered tissues, including connective tissue constructs, in various embodiments, are any suitable shape. In some embodiments, the shape is selected to mimic a particular natural tissue or organ. In further embodiments, the shape is selected to mimic a particular pathology, condition, or disease state. In some embodiments, the engineered tissues, including connective tissue constructs, have a shape that is substantially planar. In further embodiments, planar tissues have any suitable planar geometry including, by way of non-limiting examples, square, rectangle, polygon, circle, oval, or irregular. In some embodiments, the engineered tissues, including connective tissue constructs, have a shape that is substantially a sheet or a patch. In some embodiments, the engineered tissues have a shape that is substantially a tube, a ring, a disc, or a sac. In further embodiments, a sac is a rolled sheet, or a tube, with one closed end.

In some embodiments, the engineered tissues, including connective tissue constructs, are spatially confined on one or more sides by a biocompatible material. In other embodiments, the engineered tissues, including connective tissue constructs, are affixed to a surface. In further embodiments, the tissues are affixed to a biocompatible surface. In still further embodiments, a plurality of tissues are associated by affixation to a surface and spatially arranged to form an array, as described herein. In some embodiments, engineered tissues, including connective tissue constructs, are subjected to mechanical or biomechanical forces. In further embodiments, application of soluble, mechanical or biomechanical force serves to facilitate the differentiation, maturation, and development of a tissue and/or facilitate the migration, differentiation, or proliferation of cells within the tissue.

### Cells

Disclosed herein, in some embodiments, are engineered connective tissues comprising one or more types of mammalian cells. In some embodiments, the tissues include connective tissue cells. In some embodiments, the connective tissue cells are derived from multi-potent cells. In further embodiments, the connective tissue cells are derived from mesenchymal stem/stromal cells. In further embodiments, the connective tissue cells are derived from induced pluripotent stem cells. In further embodiments, the connective tissue cells are derived from embryonic stem cells. In still further embodiments, the tissues include human multi-potent cells. In still further embodiments, the tissues include human mesenchymal stem/stromal cells. In still further embodiments, the tissues include human induced pluripotent stem cells. In still further embodiments, the tissues include human embryonic stem cells.

Also disclosed herein, in some embodiments, are living, three-dimensional tissue constructs comprising multi-potent cells, wherein the multi-potent cells have been exposed to one or more differentiation signals to generate connective tissue cells or connective tissue-associated cells. In further embodiments, the tissues further include, for example, mammalian endothelial cells and/or mammalian fibroblasts.

In some embodiments, the engineered tissues include non-differentiated cells. In further embodiments, “non-differentiated cells” are cells that do not have, or have lost, the definitive tissue-specific traits of, for example, osteocytes, chondrocytes, adipose cells, fibroblasts, or endothelial cells. In some embodiments, non-differentiated cells include stem cells. In some embodiments, “stem cells” are cells that exhibit potency and self-renewal. Stem cells include, but are not limited to, totipotent cells, pluripotent cells, multi-potent cells, oligopotent cells, unipotent cells, and progenitor cells. Stem cells may be embryonic stem cells, adult stem cells, amniotic stem cells, and induced pluripotent stem cells. In yet other embodiments, the cells are a mixture of differentiated cells and non-differentiated cells. In some embodiments, the engineered tissues include mesenchymal stem/stromal cells. In further embodiments, “mesenchymal stem/stromal cells” are multi-potent cells that potentially differentiate into a variety of cell types and exhibit the properties and characteristics described further herein. In still further embodiments, the term “mesenchymal stromal cells” is used interchangeably with “mesenchymal stem/stromal cells.”

In some embodiments, the mesenchymal stem/stromal cells are human cells having multi-lineage mesenchymal differentiation potential including the capacity to differentiate into osteoblasts, adipocytes, and chondroblasts. In still further embodiments, the mesenchymal stem/stromal cells have the potential to differentiate to osteoblasts, chondroblasts, and adipocytes using standard in vitro tissue culture-differentiating conditions. In some embodiments, the mesenchymal stem/stromal cells exhibit identifiable surface antigen expression patterns. In further embodiments, the mesenchymal stem/stromal cells express the surface antigens CD105 (also known as endoglin), CD73 (also known as ecto 5′ nucleotidase) and CD90 (also known as Thy-1). In some embodiments, the mesenchymal stem/stromal cells lack expression of surface antigens specific to other cells likely to be present in mesenchymal stem cell cultures. In further embodiments, the mesenchymal stem/stromal cells lack expression of CD45 (a pan-leukocyte marker); CD34 (present on primitive hematopoietic progenitors and endothelial cells); CD14 and CD11b (prominently expressed on monocytes and macrophages); CD79a and CD19 (markers of B cells); and HLA-DR. In some embodiments, the mesenchymal stem/stromal cells exhibit adherence to plastic when maintained in standard culture conditions using tissue culture flasks. In some embodiments, the mesenchymal stem/stromal cells are human cells meeting the International Society for Cellular Therapy (ISCT) guidelines providing the most widely accepted definition of “Mesenchymal Stem Cell.” See Dominici, M. et al. Minimal criteria for defining multipotent mesenchymal stromal cells. The International Society for Cellular Therapy position statement. Cytotherapy (2006) Vol. 8, No. 4, 315-317.

In some embodiments, suitable multi-potent cells (e.g., stem cells) are derived from tissue including, by way of non-limiting example, adipose tissue, bone marrow, amniotic fluid, and umbilical tissue. In further embodiments, some or all of the stem cells are derived from mammalian lipoaspirate. In some embodiments, suitable stem cells are mesenchymal stem/stromal cells derived from mammalian adipose tissue or bone marrow. In other embodiments, some or all of the mesenchymal stem/stromal cells are derived from non-adipose, non-bone marrow tissue sources. In other embodiments, the non-adipose, non-bone marrow tissue source from which the mesenchymal stem/stromal cells are derived is selected from: blood, urine, a urologic tissue (bladder, ureter, urethra, etc.), kidney, lung, liver, stomach, intestine, trachea, esophagus, pancreas, skin, oral mucosa, dental tissue (tooth, pulp, etc.), cartilage, bone, brain, nerve, placenta, muscle tissue, omentum, mesothelium, peritoneum, lining of the nasal passages, or reproductive tissue (uterus, fallopian tube, etc.).

In some embodiments, the engineered tissues include one or more types of differentiated cells. In further embodiments, “differentiated cells” are cells with a tissue-specific phenotype consistent with, for example, a smooth muscle cell, a fibroblast, or an endothelial cell at the time of isolation, wherein tissue-specific phenotype (or the potential to display the phenotype) is maintained from the time of isolation to the time of use.

In some embodiments, any mammalian cell is suitable for further inclusion in the engineered tissues and arrays thereof. In further embodiments, the mammalian cells are, by way of non-limiting examples, contractile or muscle cells (e.g., skeletal muscle cells, cardiomyocytes, smooth muscle cells, and myoblasts), connective tissue cells (e.g., bone cells, cartilage cells, fibroblasts, and cells differentiating into bone forming cells, and chondrocytes), bone marrow cells, endothelial cells, skin cells, epithelial cells, breast cells, vascular cells, blood cells, lymph cells, neural cells, Schwann cells, gastrointestinal cells, liver cells, pancreatic cells, lung cells, tracheal cells, corneal cells, genitourinary cells, kidney cells, reproductive cells, adipose cells, parenchymal cells, pericytes, mesothelial cells, stromal cells, undifferentiated cells (e.g., embryonic cells, stem cells, and progenitor cells), endoderm-derived cells, mesoderm-derived cells, ectoderm-derived cells, and combinations thereof. In embodiments including more than one cell type, the cell types are present in many suitable ratios, examples of which are described herein.

In one embodiment, the tissues include endothelial cells. In another embodiment, the tissues include fibroblasts. In another embodiment, the tissues include endothelial cells and fibroblasts. In some embodiments, the endothelial cells are human endothelial cells. In some embodiments, suitable endothelial cells originated from tissue including, by way of non-limiting example, blood, blood vessel, lymphatic vessel, tissue of the digestive tract, tissue of the genitourinary tract, adipose tissue, tissue of the respiratory tract, tissue of the reproductive system, bone marrow, and umbilical tissue. In some embodiments, the fibroblasts are human fibroblasts. In some embodiments, suitable fibroblasts are non-vascular fibroblasts, such as dermal fibroblasts. In other embodiments, suitable fibroblasts are derived from vascular adventitia. In some embodiments, some or all of the cells are derived from mammalian lipoaspirate. In further embodiments, some or all of the cells are cultured from the stromal vascular fraction of mammalian lipoaspirate.

In various embodiments, the cell types and/or source of the cells are selected, configured, treated, or modulated based on a specific research goal or objective. In some embodiments, one or more specific cell types are selected, configured, treated, or modulated to facilitate investigation of a particular disease or condition. In some embodiments, one or more specific cell types are selected, configured, treated, or modulated to facilitate investigation of a disease or a condition of a particular subject. In some embodiments, one or more specific cell types are derived from two or more distinct human donors. In some embodiments, one or more specific cell types are derived from a particular vertebrate subject. In further embodiments, one or more specific cell types are derived from a particular mammalian subject. In still further embodiments, one or more specific cell types are derived from a particular human subject.

### Methods of Culturing Cells

The cell types used in the engineered tissues of the invention may be cultured in any manner known in the art. Methods of cell and tissue culturing are known in the art, and are described, for example, in Cell & Tissue Culture: Laboratory Procedures; Freshney (1987), Culture of Animal Cells: A Manual of Basic Techniques, the contents of which are incorporated herein by reference for such information. General mammalian cell culture techniques, cell lines, and cell culture systems that may be used in conjunction with the present invention are also described in Doyle, A., Griffiths, J. B., Newell, D. G., (eds.) Cell and Tissue Culture: Laboratory Procedures, Wiley (1998), the contents of which are incorporated herein by reference for such information.

Appropriate growth conditions for mammalian cells in culture are well known in the art. Cell culture media generally include essential nutrients and, optionally, additional elements such as growth factors, salts, minerals, vitamins, etc., that may be selected according to the cell type(s) being cultured. Particular ingredients may be selected to enhance cell growth, differentiation, secretion of specific proteins, etc. In general, standard growth media include Dulbecco's Modified Eagle Medium, low glucose (DMEM), with 110 mg/L pyruvate and glutamine, supplemented with 10-20% fetal bovine serum (FBS), calf serum, or human serum and 100 U/ml penicillin, 0.1 mg/ml streptomycin are appropriate as are various other standard media well known to those in the art. Preferably cells are cultured under sterile conditions in an atmosphere of 1-21% O₂ and preferably 3-5% CO₂, at a temperature at or near the body temperature of the animal of origin of the cell. For example, human cells are preferably cultured at approximately 37° C. With regard to mesenchymal stem/stromal cells, suitable culture media includes basal media containing 5-10% (v:v) fetal bovine serum in low glucose DMEM supplemented with L-glutamine. Optionally, mesenchymal stem/stromal cells are cultured and expanded in conditions wherein the oxygen tension is less than 21% oxygen (equivalent to atmospheric oxygen tension). In some embodiments, the cells are cultured at 3-5% oxygen conditions.

The cells can also be cultured with cellular differentiation agents to induce differentiation of the cell along a desired line. For example, in some embodiments, stem cells are incubated in contact with differentiation media to produce a range of cell types. Many types of differentiation media are suitable. In various embodiments stem cells are incubated in contact with differentiation media including, by way of non-limiting examples, osteogenic differentiation media, chondrogenic differentiation media, adipogenic differentiation media, neural differentiation media, cardiomyocyte differentiation media, and enterocyte differentiation media (e.g., intestinal epithelium). With regard to mesenchymal stem/stromal cells, in some embodiments, the cells are incubated in contact with differentiation media including, by way of non-limiting examples, osteogenic differentiation media, chondrogenic differentiation media, or adipogenic differentiation media.

Additionally, cells can be cultured with growth factors, cytokines, etc. In some embodiments, the term “growth factor” refers to a protein, a polypeptide, or a complex of polypeptides, including cytokines, that are produced by a cell and which can affect itself and/or a variety of other neighboring or distant cells. Typically growth factors affect the growth and/or differentiation of specific types of cells, either developmentally or in response to a multitude of physiological or environmental stimuli. Some, but not all, growth factors are hormones. Exemplary growth factors are insulin, insulin-like growth factor (IGF), nerve growth factor (NGF), vascular endothelial growth factor (VEGF), keratinocyte growth factor (KGF), fibroblast growth factors (FGFs), including basic FGF (bFGF), platelet-derived growth factors (PDGFs), including PDGF-AA and PDGF-AB, hepatocyte growth factor (HGF), transforming growth factor alpha (TGF-α), transforming growth factor beta (TGF-β), including TGFβ1 and TGFβ3, epidermal growth factor (EGF), granulocyte-macrophage colony-stimulating factor (GM-CSF), granulocyte colony-stimulating factor (G-CSF), interleukin-6 (IL-6), IL-8, and the like. Growth factors are discussed in, among other places, Molecular Cell Biology, Scientific American Books, Darnell et al., eds., 1986; Principles of Tissue Engineering, 2d ed., Lanza et al., eds., Academic Press, 2000. The skilled artisan will understand that any and all culture-derived growth factors in the conditioned media described herein are within the scope of the invention.

### Bio-Ink and Multicellular Aggregates

Disclosed herein, in certain embodiments, are tissues, including connective tissue constructs, arrays thereof, and methods that comprise bioprinted cells. In some embodiments, cells are bioprinted by depositing or extruding bio-ink from a bioprinter. In some embodiments, “bio-ink” includes liquid, semi-solid, or solid compositions comprising a plurality of cells.

In some embodiments, bio-ink comprises liquid or semi-solid cell solutions, cell suspensions, or cell concentrations. In some embodiments, bio-ink comprises semi-solid or solid multicellular aggregates or multicellular bodies. In further embodiments, the bio-ink is produced by 1) mixing a plurality of cells or cell aggregates and a biocompatible liquid or gel in a pre-determined ratio to result in bio-ink, and 2) compacting the bio-ink to produce the bio-ink with a desired cell density and viscosity. In some embodiments, the compacting of the bio-ink is achieved by centrifugation, tangential flow filtration (“TFF”), or a combination thereof. In some embodiments, the compacting of the bio-ink results in a composition that is extrudable, allowing formation of multicellular aggregates or multicellular bodies. In some embodiments, “extrudable” means able to be shaped by forcing (e.g., under pressure) through a nozzle or orifice (e.g., one or more holes or tubes). In some embodiments, the compacting of the bio-ink results from growing the cells to a suitable density. The cell density necessary for the bio-ink will vary with the cells being used and the tissue or organ being produced. In some embodiments, the cells of the bio-ink are cohered and/or adhered. In some embodiments, “cohere,” “cohered,” and “cohesion” refer to cell-cell adhesion properties that bind cells, multicellular aggregates, multicellular bodies, and/or layers thereof. In further embodiments, the terms are used interchangeably with “fuse,” “fused,” and “fusion.” In some embodiments, the bio-ink additionally comprises support material, cell culture medium, extracellular matrix (or components thereof), cell adhesion agents, cell death inhibitors, anti-apoptotic agents, anti-oxidants, extrusion compounds, and combinations thereof.

In various embodiments, the cells are any suitable cell. In further various embodiments, the cells are vertebrate cells, mammalian cells, human cells, or combinations thereof. In some embodiments, the cells include stem cells. In further embodiments, the stem cells are human stem cells. In some embodiments, the cells include mesenchymal stem/stromal cells. In further embodiments, the mesenchymal stem/stromal cells are human mesenchymal stem/stromal cells. In some embodiments, the type of cell used in a method disclosed herein depends on the type of construct or tissue being produced. In some embodiments, the bio-ink comprises one type of cell. In some embodiments, the bio-ink comprises more than one type of cell.

### Cell Culture Media

In some embodiments, the bio-ink comprises a cell culture medium. The cell culture medium is any suitable medium. In various embodiments, suitable cell culture media include, by way of non-limiting examples, Dulbecco's Phosphate Buffered Saline, Earle's Balanced Salts, Hanks' Balanced Salts, Tyrode's Salts, Alsever's Solution, Gey's Balanced Salt Solution, Kreb's-Henseleit Buffer Modified, Kreb's-Ringer Bicarbonate Buffer, Puck's Saline, Dulbecco's Modified Eagle's Medium, Dulbecco's Modified Eagle's Medium/Nutrient F-12 Ham, Nutrient Mixture F-10 Ham (Ham's F-10), Medium 199, Minimum Essential Medium Eagle, RPMI-1640 Medium, Ames' Media, BGJb Medium (Fitton-Jackson Modification), Click's Medium, CMRL-1066 Medium, Fischer's Medium, Glascow Minimum Essential Medium (GMEM), Iscove's Modified Dulbecco's Medium (IMDM), L-15 Medium (Leibovitz), McCoy's 5A Modified Medium, NCTC Medium, Swim's S-77 Medium, Waymouth Medium, William's Medium E, or combinations thereof. In some embodiments, the cell culture medium is modified or supplemented. In some embodiments, the cell culture medium further comprises albumin, selenium, transferrins, fetuins, sugars, amino acids, vitamins, growth factors, cytokines, hormones, antibiotics, lipids, lipid carriers, cyclodextrins, or combinations thereof. In some embodiments, the cell culture medium is a stem cell differentiation medium. In further embodiments, the stem cell differentiation medium is, by way of non-limiting examples, an osteogenic differentiation medium, a chondrogenic differentiation medium, or an adipogenic differentiation medium.

### Extracellular Matrix

In some embodiments, the bio-ink further comprises one or more components of an extracellular matrix or derivatives thereof. In some embodiments, “extracellular matrix” includes proteins that are produced by cells and transported out of the cells into the extracellular space, where they may serve as a support to hold tissues together, to provide tensile strength, and/or to facilitate cell signaling. Examples, of extracellular matrix components include, but are not limited to, collagen, fibronectin, laminin, hyaluronates, elastin, and proteoglycans. For example, the multicellular aggregates may contain various ECM proteins (e.g., gelatin, fibrinogen, fibrin, collagen, fibronectin, laminin, elastin, and/or proteoglycans). The ECM components or derivatives of ECM components can be added to the cell paste used to form the multicellular aggregate. The ECM components or derivatives of ECM components added to the cell paste can be purified from a human or animal source, or produced by recombinant methods known in the art. Alternatively, the ECM components or derivatives of ECM components can be naturally secreted by the cells in the elongate cellular body, or the cells used to make the elongate cellular body can be genetically manipulated by any suitable method known in the art to vary the expression level of one or more ECM components or derivatives of ECM components and/or one or more cell adhesion molecules or cell-substrate adhesion molecules (e.g., selectins, integrins, immunoglobulins, and adherins). The ECM components or derivatives of ECM components may promote cohesion of the cells in the multicellular aggregates. For example, gelatin and/or fibrinogen can suitably be added to the cell paste, which is used to form multicellular aggregates. The fibrinogen can then be converted to fibrin by the addition of thrombin.

In some embodiments, the bio-ink further comprises an agent that encourages cell adhesion.

In some embodiments, the bio-ink further comprises an agent that inhibits cell death (e.g., necrosis, apoptosis, or autophagocytosis). In some embodiments, the bio-ink further comprises an anti-apoptotic agent. Agents that inhibit cell death include, but are not limited to, small molecules, antibodies, peptides, peptibodies, or combination thereof. In some embodiments, the agent that inhibits cell death is selected from: anti-TNF agents, agents that inhibit the activity of an interleukin, agents that inhibit the activity of an interferon, agents that inhibit the activity of an GCSF (granulocyte colony-stimulating factor), agents that inhibit the activity of a macrophage inflammatory protein, agents that inhibit the activity of TGF-B (transforming growth factor B), agents that inhibit the activity of an MMP (matrix metalloproteinase), agents that inhibit the activity of a caspase, agents that inhibit the activity of the MAPK/JNK signaling cascade, agents that inhibit the activity of a Src kinase, agents that inhibit the activity of a JAK (Janus kinase), or a combination thereof. In some embodiments, the bio-ink comprises an anti-oxidant.

### Extrusion Compounds

In some embodiments, the bio-ink further comprises an extrusion compound (i.e., a compound that modifies the extrusion properties of the bio-ink). Examples of extrusion compounds include, but are not limited to gels, hydrogels, surfactant polyols (e.g., Pluronic F-127 or PF-127), thermo-responsive polymers, UV light-responsive polymers, hyaluronates, alginates, extracellular matrix components (and derivatives thereof), gelatins, collagens, peptide hydrogels, other biocompatible natural or synthetic polymers, nanofibers, and self-assembling nanofibers.

Gels, sometimes referred to as jellies, have been defined in various ways. For example, the United States Pharmacopoeia defines gels as semisolid systems consisting of either suspensions made up of small inorganic particles or large organic molecules interpenetrated by a liquid. Gels include a single-phase or a two-phase system. A single-phase gel consists of organic macromolecules distributed uniformly throughout a liquid in such a manner that no apparent boundaries exist between the dispersed macromolecules and the liquid. Some single-phase gels are prepared from synthetic macromolecules (e.g., carbomer) or from natural gums (e.g., tragacanth). In some embodiments, single-phase gels are generally aqueous, but will also be made using alcohols and oils. Two-phase gels consist of a network of small discrete particles.

Gels can also be classified as being hydrophobic or hydrophilic. In certain embodiments, the base of a hydrophobic gel consists of liquid paraffin with polyethylene or fatty oils gelled with colloidal silica, or aluminum or zinc soaps. In contrast, the base of hydrophobic gels usually consists of water, glycerol, or propylene glycol gelled with a suitable gelling agent (e.g., tragacanth, starch, cellulose derivatives, carboxyvinylpolymers, and magnesium-aluminum silicates). In certain embodiments, the rheology of the compositions or devices disclosed herein is pseudo plastic, plastic, thixotropic, or dilatant.

Suitable hydrogels include those derived from collagen, hyaluronate, fibrin, alginate, agarose, chitosan, and combinations thereof. In other embodiments, suitable hydrogels are synthetic polymers. In further embodiments, suitable hydrogels include those derived from poly(acrylic acid) and derivatives thereof, poly(ethylene oxide) and copolymers thereof, poly(vinyl alcohol), polyphosphazene, and combinations thereof. In various specific embodiments, the confinement material is selected from: hydrogel, NovoGel™, agarose, alginate, gelatin, Matrigel™, hyaluronan, poloxamer, peptide hydrogel, poly(isopropyl n-polyacrylamide), polyethylene glycol diacrylate (PEG-DA), hydroxyethyl methacrylate, polydimethylsiloxane, polyacrylamide, poly(lactic acid), silicon, silk, peptide hydrogels, or combinations thereof.

In some embodiments, hydrogel-based extrusion compounds are thermoreversible gels (also known as thermo-responsive gels or thermogels). In some embodiments, a suitable thermoreversible hydrogel is not a liquid at room temperature. In specific embodiments, the gelation temperature (Tgel) of a suitable hydrogel is about 10° C., about 15° C., about 20° C., about 25° C., about 30° C., about 35° C., and about 40° C., including increments therein. In certain embodiments, the Tgel of a suitable hydrogel is about 10° C. to about 25° C. In some embodiments, the bio-ink (e.g., comprising hydrogel, one or more cell types, and other additives, etc.) described herein is not a liquid at room temperature. In specific embodiments, the gelation temperature (Tgel) of a bio-ink described herein is about 10° C., about 15° C., about 20° C., about 25° C., about 30° C., about 35° C., and about 40° C., including increments therein. In certain embodiments, the Tgel of a bio-ink described herein is about 10° C. to about 25° C.

Polymers composed of polyoxypropylene and polyoxyethylene form thermoreversible gels when incorporated into aqueous solutions. These polymers have the ability to change from the liquid state to the gel state at temperatures that can be maintained in a bioprinter apparatus. The liquid state-to-gel state phase transition is dependent on the polymer concentration and the ingredients in the solution.

Poloxamer 407 (Pluronic F-127 or PF-127) is a nonionic surfactant composed of polyoxyethylene-polyoxypropylene copolymers. Other poloxamers include 188 (F-68 grade), 237 (F-87 grade), 338 (F-108 grade). Aqueous solutions of poloxamers are stable in the presence of acids, alkalis, and metal ions. PF-127 is a commercially available polyoxyethylene-polyoxypropylene triblock copolymer of general formula E106 P70 E106, with an average molar mass of 13,000. The polymer can be further purified by suitable methods that will enhance gelation properties of the polymer. It contains approximately 70% ethylene oxide, which accounts for its hydrophilicity. It is one of the series of poloxamer ABA block copolymers. PF-127 has good solubilizing capacity, low toxicity and is, therefore, considered a suitable extrusion compound.

In some embodiments, the viscosity of the hydrogels and bio-inks presented herein is measured by any means described. For example, in some embodiments, an LVDV-II+CP Cone Plate Viscometer and a Cone Spindle CPE-40 is used to calculate the viscosity of the hydrogels and bio-inks. In other embodiments, a Brookfield (spindle and cup) viscometer is used to calculate the viscosity of the hydrogels and bio-inks. In some embodiments, the viscosity ranges referred to herein are measured at room temperature. In other embodiments, the viscosity ranges referred to herein are measured at body temperature (e.g., at the average body temperature of a healthy human).

In further embodiments, the hydrogels and/or bio-inks are characterized by having a viscosity of between about 500 and 1,000,000 centipoise, between about 750 and 1,000,000 centipoise; between about 1000 and 1,000,000 centipoise; between about 1000 and 400,000 centipoise; between about 2000 and 100,000 centipoise; between about 3000 and 50,000 centipoise; between about 4000 and 25,000 centipoise; between about 5000 and 20,000 centipoise; or between about 6000 and 15,000 centipoise.

In some embodiments, the bio-ink comprises cells and extrusion compounds suitable for continuous bioprinting. In specific embodiments, the bio-ink has a viscosity of about 1500 mPa·s. A mixture of Pluronic F-127 and cellular material may be suitable for continuous bioprinting. Such a bio-ink may be prepared by dissolving Pluronic F-127 powder by continuous mixing in cold (4° C.) phosphate buffered saline (PBS) over 48 hours to 30% (w/v). Pluronic F-127 may also be dissolved in water. Cells may be cultivated and expanded using standard sterile cell culture techniques. The cells may be pelleted at 200 g for example, and re-suspended in the 30% Pluronic F-127 and aspirated into a reservoir affixed to a bioprinter where it can be allowed to solidify at a gelation temperature from about 10 to about 25° C. Gelation of the bio-ink prior to bioprinting is optional. The bio-ink, including bio-ink comprising Pluronic F-127 can be dispensed as a liquid.

In various embodiments, the concentration of Pluronic F-127 can be any value with suitable viscosity and/or cytotoxicity properties. A suitable concentration of Pluronic F-127 may also be able to support weight while retaining its shape when bioprinted. In some embodiments, the concentration of Pluronic F-127 is about 10%, about 15%, about 20%, about 25%, about 30%, about 35%, about 40%, about 45%, or about 50%. In some embodiments, the concentration of Pluronic F-127 is between about 30% and about 40%, or between about 30% and about 35%.

In some embodiments, the non-cellular components of the bio-ink (e.g., extrusion compounds, etc.) are removed prior to use. In further embodiments, the non-cellular components are, for example, hydrogels, surfactant polyols, thermo-responsive polymers, hyaluronates, alginates, collagens, or other biocompatible natural or synthetic polymers. In still further embodiments, the non-cellular components are removed by physical, chemical, or enzymatic means. In some embodiments, a proportion of the non-cellular components remain associated with the cellular components at the time of use.

In some embodiments, the cells are pre-treated to increase cellular interaction. For example, cells may be incubated inside a centrifuge tube after centrifugation in order to enhance cell-cell interactions prior to shaping the bio-ink.

### Exemplary Cell Ratios

In some embodiments, the bio-ink comprises multicellular bodies, which further comprise mesenchymal stem/stromal cells. In further embodiments, the bio-ink comprises multicellular bodies, which further comprise mesenchymal stem/stromal cells and one or more other cell types. In still further embodiments, the bio-ink comprises multicellular bodies, which further comprise mesenchymal stem/stromal cells and endothelial cells, fibroblasts, or both endothelial cells and fibroblasts.

In some embodiments, bio-ink is prepared with any suitable ratio of mesenchymal stem/stromal cells to other cell types. For example, in some embodiments, bio-ink is prepared with a ratio of mesenchymal stem/stromal cells to endothelial cells between about 5:1 to about 20:1. In various further embodiments, the ratio of mesenchymal stem/stromal cells to endothelial cells is about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, or about 20:1, including increments therein. In still further embodiments, the ratio of mesenchymal stem/stromal cells to endothelial cells is about 9:1.

By way of further example, in some embodiments, bio-ink is prepared with a ratio of mesenchymal stem/stromal cells to fibroblasts between about 5:1 to about 20:1. In various further embodiments, the ratio of mesenchymal stem/stromal cells to fibroblasts is about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, or about 20:1, including increments therein. In still further embodiments, the ratio of mesenchymal stem/stromal cells to fibroblasts is about 9:1.

### Self-Sorting of Cells

In some embodiments, multicellular aggregates used to form the construct or tissue comprises all cell types to be included in the engineered tissue (e.g., endothelial cells, smooth muscle cells, fibroblasts, etc.); in such an example each cell type migrates to an appropriate position (e.g., during maturation) to form the engineered tissue, such as a connective tissue construct. In other embodiments, the multicellular aggregates used to form the structure comprises fewer than all the cell types to be included in the engineered tissue. In some embodiments, cells of each type are uniformly distributed within a multicellular aggregates, or region or layer of the tissue. In other embodiments, cells of each type localize to particular regions within a multicellular aggregate or layers or regions of the tissue.

### Differentiation Signals

In some embodiments, disclosed herein are engineered tissues and arrays thereof comprising connective tissue cells cohered to one another, wherein the connective tissue cells are derived from multi-potent cells. Also disclosed herein are engineered tissues and arrays thereof comprising multi-potent cells cohered to one another, wherein the multi-potent cells have been exposed to one or more differentiation signals. In various embodiments, the multi-potent cells have been exposed to, for example, 1, 2, 3, 4, 5, 6, 7, 8, 9, 10, 11, 12, 13, 14, 15, 16, 17, 18, 19, 20 or more differentiation signals.

### Types of Differentiation Signals

In some embodiments, one or more differentiation signals include mechanical, biomechanical, or physical signals, including combinations thereof. In further embodiments, mechanical, biomechanical, or physical signals include, by way of non-limiting examples, stretching, bending, compressing, increased atmospheric pressure, shear force caused by fluid flow, and combinations thereof.

In some embodiments, one or more differentiation signals include chemical or biochemical signals, including combinations thereof. In further embodiments, a chemical or biochemical signal includes, by way of non-limiting examples, exposure to a nutrient, hormone, growth factor, or chemical agent.

In some embodiments, one or more differentiation signals include incubation in differentiation media. In further embodiments, a differentiation media supports, facilitates, and/or triggers differentiation of in vitro cultures of stem cells toward one or more specific phenotypes. In still further embodiments, a differentiation media supports, facilitates, and/or triggers differentiation of in vitro cultures of mesenchymal stem/stromal cells toward one or more connective tissue phenotypes via osteogenesis, chondrogenesis, and/or adipogenesis.

Exposure to one or more differentiation signals has a wide range of suitable durations. In various embodiments, stem cells are exposed to one or more differentiation signal for, by way of non-limiting examples, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more seconds, including increments therein. In various embodiments, stem cells are exposed to one or more differentiation signal for, by way of non-limiting examples, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 or more minutes, including increments therein. In various further embodiments, stem cells are exposed to one or more differentiation signal for, by way of non-limiting examples, 1, 2, 3, 4, 5, 6, 7, 8, 9, 10, 11, 12, 13, 14, 15, 16, 17, 18, 19, 20, 21, 22, 23, 24 or more hours, including increments therein. In various further embodiments, stem cells are exposed to one or more differentiation signal for, by way of non-limiting examples, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more days, including increments therein.

### Time Periods for Exposure to Differentiation Signals

Many time periods, relative to fabrication of an engineered tissue construct are suitable for exposure of multi-potent cells (e.g., stem cells) to one or more differentiation signals. In some embodiments, stem cells are exposed to one or more differentiation signals before fabrication of a tissue construct. In further embodiments, cells are exposed one or more differentiation signals in cell culture prior to creation of bio-ink or before deposition of cells/bio-ink to form a tissue construct (e.g., pre-deposition). In still further embodiments a pre-deposition exposure to one or more differentiation signals is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more days before deposition of cells/bio-ink to form a tissue construct. In some embodiments, a pre-deposition exposure to one or more differentiation signals is about 5 to about 21 days before deposition of cells/bio-ink to form a tissue construct. In some embodiments, a pre-deposition exposure to one or more differentiation signals is about 5 to about 0 days before deposition of cells/bio-ink to form a tissue construct.

In some embodiments, stem cells are exposed to one or more differentiation signals around the time of fabrication of a tissue construct and/or during fabrication. In further embodiments, cells are exposed one or more differentiation signals around the time of and/or during creation of bio-ink. In further embodiments, cells are exposed one or more differentiation signals around the time of and/or during deposition of cells/bio-ink to form a tissue construct (e.g., peri-deposition). In still further embodiments, a peri-deposition exposure to one or more differentiation signals is within about 1, 2, 3, 4, 5, 6, 7, 8, 9, 10, 11, 12, 13, 14, 15, 16, 17, 18, 19, 20, 21, 22, 23, 24 or more hours of deposition of cells/bio-ink to form a tissue construct. In still further embodiments, a peri-deposition exposure to one or more differentiation signals is within about 1, 2, 3, 4, 5, 6, 7, 8, 9, 10 or more days of deposition of cells/bio-ink to form a tissue construct. In some embodiments, a peri-deposition exposure to one or more differentiation signals is within about 5 days of deposition of cells/bio-ink to form a tissue construct. In some embodiments, a pre-deposition exposure to one or more differentiation signals is within about 2 days of deposition of cells/bio-ink to form a tissue construct.

In some embodiments, stem cells are exposed to one or more differentiation signals after fabrication of a tissue construct. In further embodiments, cells are exposed one or more differentiation signals in a culture after deposition of cells/bio-ink to form a tissue construct (e.g., post-deposition). In still further embodiments a post-deposition exposure to one or more differentiation signals is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more days after deposition of cells/bio-ink to form a tissue construct. In some embodiments, a post-deposition exposure to one or more differentiation signals is about 1 to about 21 days after deposition of cells/bio-ink to form a tissue construct. In some embodiments, a post-deposition exposure to one or more differentiation signals is about 5 to about 0 days after deposition of cells/bio-ink to form a tissue construct.

In some embodiments, some portion of the mesenchymal stem/stromal cells in bio-ink and/or a connective tissue construct are characterized by partial or complete differentiation toward a cell type present in mammalian connective tissue including, by way of non-limiting examples, osteocytes, chondrocytes, and adipocytes. In various embodiments, 1, 5, 10, 20, 30, 40, 50, 60, 70 80, 90, 95, 98, 99, or 100 percent of the mesenchymal stem/stromal cells exhibit some degree of differentiation.

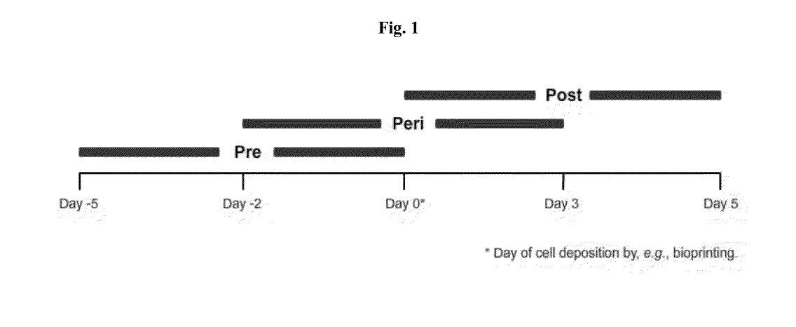

Referring to FIG. 1, in a particular embodiment, a variety of time periods are suitable for exposure of mesenchymal stem/stromal cells to osteogenic differentiation media. In this embodiment, three suitable time periods are defined relative to fabrication of a tissue construct by deposition of cells via, e.g., a bioprinter. Further in this embodiment, mesenchymal stem/stromal cells are optionally exposed to osteogenic differentiation media pre-deposition, peri-deposition, and/or post-deposition. In this case, a pre-deposition time period extends from 5 days before deposition to the day of deposition; a peri-deposition time period extends from 2 days before deposition to 3 days after deposition; and a post-deposition time period extends from the day of deposition to 5 days after deposition.

In some embodiments, disclosed herein are engineered connective tissue constructs, and arrays thereof comprising mesenchymal stem/stromal cells cohered to one another, wherein the mesenchymal stem/stromal cells have been exposed to one or more differentiation signals to provide a living, three-dimensional connective tissue construct.

In some embodiments, the connective tissue is by way of non-limiting examples, bone, cartilage, tendon, ligament, and combinations thereof. In some embodiments, the connective tissue a compound tissue including, for example, bone, cartilage, tendon, ligament, combinations thereof and a non-connective tissue. In further embodiments, cartilage and bone may be combined in a layered tissue to form connective tissue for use in joint repair. In further embodiments, engineered connective tissue constructs may be designed to be compatible with implantable medical devices or prosthetics to enhance engraftment or function of the device or prosthetic. In some embodiments, ligaments may be engineered to include osteoid tissue on one or both ends to aide in surgical delivery or engraftment, or to enhance function after delivery. In still further embodiments, tendons may be engineered with osteoid tissue on one end and/or muscle tissue on the opposing end, to aide in surgical delivery or engraftment, or to enhance function after delivery.

### Assessment of Differentiation

A wide variety of techniques and methods are suitable for assessment of multi-potent cell (e.g., stem cell) differentiation toward a specific tissue phenotype. In some embodiments, microscopy and staining is used to assess differentiation by identifying specific chemical substances, cell surface antigens cell organelles, cellular structures, and/or cell populations. With regard to assessment of differentiation of mesenchymal stem/stromal cells toward a connective tissue phenotype, in some embodiments, Alizarin Red S (staining calcium crystals) and/or Von Kossa (staining calcium phosphate deposits) are utilized to identify and optionally quantify osteogenesis. By way of further example, elevated levels of alkaline phosphatase indicates active bone formation occurring as this enzyme is a byproduct of osteoblast activity; therefore, in some embodiments, alkaline phosphatase staining is used to detect differentiation toward a bone phenotype. In some embodiments, Enzyme-linked immunosorbent assay (ELISA) is used to assess differentiation by identifying specific chemical substances, cell surface antigens, cell organelles, cellular structures, and/or cell populations. With regard to assessment of differentiation of mesenchymal stem/stromal cells toward a connective tissue phenotype, in some embodiments, ELISA for osteopontin (an extracellular structural protein expressed in osteoblasts) is utilized to identify and optionally quantify osteogenesis.

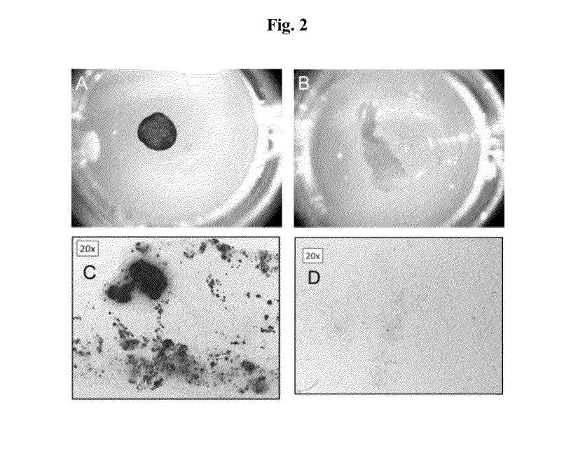

Referring to FIGS. 2A and 2B, in a particular embodiment, mesenchymal stem cell-containing constructs were bioprinted and cultured in either osteogenic differentiation medium or only basal mesenchymal stem cell culture media. In situ alkaline phosphatase staining of bioprinted constructs was utilized to detect osteoblast activity. FIG. 2A illustrates expression of alkaline phosphatase in constructs exposed to osteogenic differentiation medium. Whereas FIG. 2B illustrates little or no expression of alkaline phosphatase in constructs exposed only to basal mesenchymal stem cell culture media.

Referring to FIGS. 2C and 2D, in a particular embodiment, mesenchymal stem cell-containing constructs were bioprinted and cultured in either osteogenic differentiation medium or only basal mesenchymal stem cell culture media immediately post-printing. Calcium deposits were identified by Alizarin Red S staining FIG. 2C illustrates deposition of calcium in constructs exposed to osteogenic differentiation medium. Whereas FIG. 2D illustrates little or no calcium present in constructs exposed only to basal mesenchymal stem cell culture media.

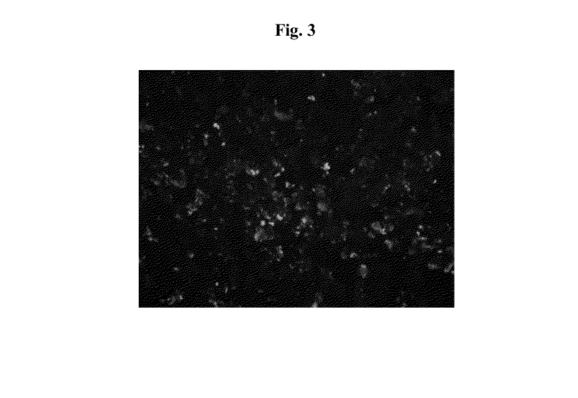

Referring to FIG. 3, in a particular embodiment, mesenchymal stem/stromal cells were cultured and used to produce bio-ink, which was bioprinted to form tissue constructs. After 5 days of post-print incubation in differentiation media, the resulting tissue was tissue sectioned, formalin-fixed, and paraffin-embedded. Immunofluorescence staining of the constructs for expression of osteopontin was performed. The illustrated response is indicative of mesenchymal stem cell differentiation and osteogenesis.

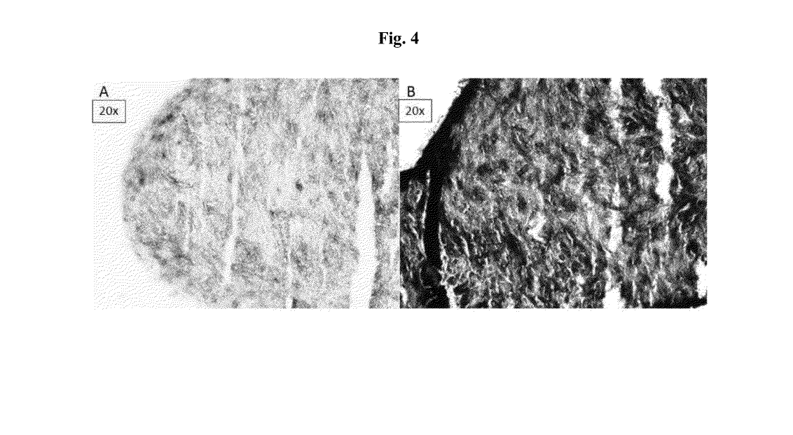

Referring to FIGS. 4A and 4B, in a particular embodiment, mesenchymal stem cell-containing constructs were bioprinted and cultured in either osteogenic differentiation medium or only basal mesenchymal stem cell culture media. Histological alkaline phosphatase staining of bioprinted constructs was utilized to detect osteoblast activity. FIG. 4A illustrates little or no expression of alkaline phosphatase in constructs exposed only to basal mesenchymal stem cell culture media. Whereas FIG. 4B illustrates expression of alkaline phosphatase in constructs exposed to osteogenic differentiation medium.

### Pre-Formed Scaffold

In some embodiments, disclosed herein are engineered tissues, including connective tissue constructs, and arrays thereof that are free or substantially free of any pre-formed scaffold. In further embodiments, “scaffold” refers to synthetic scaffolds such as polymer scaffolds and porous hydrogels, non-synthetic scaffolds such as pre-formed extracellular matrix layers and decellularized tissues, and any other type of pre-formed scaffold that is integral to the physical structure of the engineered tissue and/or organ and not removed from the tissue and/or organ.

In some embodiments, the engineered tissues, including connective tissue constructs, and arrays thereof do not utilize any pre-formed scaffold, e.g., for the formation of the tissue, any layer of the tissue, or formation of the tissue's shape. As a non-limiting example, the engineered tissues of the present invention do not utilize any pre-formed, synthetic scaffolds such as polymer scaffolds, pre-formed extracellular matrix layers, or any other type of pre-formed scaffold. In some embodiments, the engineered tissues are substantially free of any pre-formed scaffolds. In further embodiments, the cellular components of the tissues contain a detectable, but trace or trivial amount of scaffold, e.g., less than 2.0% of the total composition, less than 1.0% of the total composition, less than 0.5% of the total composition, or less than 0.1% of the total composition. In still further embodiments, trace or trivial amounts of scaffold are insufficient to affect long-term behavior of the tissue, or array thereof, or interfere with its primary biological function. In additional embodiments, scaffold components are removed post-printing, by physical, chemical, or enzymatic methods, yielding an engineered tissue that is free or substantially-free of scaffold components.

In some embodiments, the engineered tissues free, or substantially free, of pre-formed scaffold disclosed herein are in stark contrast to those developed with certain other methods of tissue engineering in which a scaffolding material is first formed, and then cells are seeded onto the scaffold, and subsequently the cells proliferate to fill and take the shape of the scaffold for example. In one aspect, the methods of bioprinting described herein allow production of viable and useful tissues that are substantially free of pre-formed scaffold. In another aspect, the cells of the invention are, in some embodiments, held in a desired three-dimensional shape using a confinement material. The confinement material is distinct from a scaffold at least in the fact that the confinement material is temporary and/or removable from the cells and/or tissue.

### Arrays

In some embodiments, disclosed herein are arrays of engineered tissues, including connective tissue constructs. In some embodiments, an “array” is a scientific tool including an association of multiple elements spatially arranged to allow a plurality of tests to be performed on a sample, one or more tests to be performed on a plurality of samples, or both. In some embodiments, the arrays are adapted for, or compatible with, screening methods and devices, including those associated with high-throughput screening. In further embodiments, an array allows a plurality of tests to be performed simultaneously. In further embodiments, an array allows a plurality of samples to be tested simultaneously. In some embodiments, the arrays are cellular microarrays. In further embodiments, a cellular microarray is a laboratory tool that allows for the multiplex interrogation of living cells on the surface of a solid support. In other embodiments, the arrays are tissue microarrays. In further embodiments, tissue microarrays include a plurality of separate tissues or tissue samples assembled in an array to allow the performance of multiple biochemical, metabolic, molecular, or histological analyses.

In some embodiments, the engineered tissues, including connective tissue constructs, each exist in a well of a biocompatible multi-well container. In some embodiments, each tissue is placed into a well. In other embodiments, each tissue is bioprinted into a well. In further embodiments, the wells are coated. In various further embodiments, the wells are coated with one or more of: a biocompatible hydrogel, one or more proteins, one or more chemicals, one or more peptides, one or more antibodies, and one or more growth factors, including combinations thereof. In some embodiments, the wells are coated with NovoGel™. In other embodiments, the wells are coated with agarose. In some embodiments, each tissue exists on a porous, biocompatible membrane within a well of a biocompatible multi-well container.

In some embodiments, the engineered tissues, including connective tissue constructs, are constrained by a biocompatible surface on one or more sides. In further embodiments, the engineered tissues, including connective tissue constructs, are held in an array configuration by being constrained by a biocompatible surface on one or more sides. In still further embodiments, the tissue is constrained by a biocompatible surface on 1, 2, 3, 4, or more sides. In some embodiments, the engineered tissues, including connective tissue constructs, are affixed to a biocompatible surface on one or more sides.

In some embodiments, the biocompatible surface is any surface that does not pose a significant risk of injury or toxicity to the tissue or an organism contacting the tissue. In further embodiments, the biocompatible surface is any surface suitable for traditional tissue culture methods. Suitable biocompatible surfaces include, by way of non-limiting examples, treated plastics, membranes, porous membranes, coated membranes, coated plastics, metals, coated metals, glass, and coated glass, wherein suitable coatings include hydrogels, ECM components, chemicals, proteins, etc.

In some embodiments, affixation of an engineered tissue to a biocompatible surface on one or more sides facilitates subjecting the tissue to mechanical or biomechanical forces. In further embodiments, the engineered tissues, including connective tissue constructs, are subjected to mechanical or biomechanical forces. In various embodiments, the engineered tissues are subjected to mechanical or biomechanical forces on 1, 2, 3, 4, or more sides.

In some embodiments, the arrays of engineered tissues, including connective tissue constructs, comprise an association of two or more elements. In various embodiments, the arrays comprise an association of 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, or 500 elements, including increments therein. In further embodiments, each element comprises one or more cells, multicellular aggregates, tissues, organs, or combinations thereof.

In some embodiments, the arrays of engineered tissues, including connective tissue constructs, comprise multiple elements spatially arranged in a pre-determined pattern. In further embodiments, the pattern is any suitable spatial arrangement of elements. In various embodiments, patterns of arrangement include, by way of non-limiting examples, a two-dimensional grid, a three-dimensional grid, one or more lines, arcs, or circles, a series of rows or columns, and the like. In further embodiments, the pattern is chosen for compatibility with high-throughput biological assay or screening methods or devices.

In various embodiments, the cell types and/or source of the cells used to fabricate one or more tissues in an array are selected based on a specific research goal or objective. In further various embodiments, the specific tissues in an array are selected based on a specific research goal or objective. In some embodiments, one or more specific engineered tissues are included in an array to facilitate investigation of a particular disease or condition. In some embodiments, one or more specific engineered tissues are included in an array to facilitate investigation of a disease or a condition of a particular subject. In further embodiments, one or more specific engineered tissues within the array are generated with one or more cell types derived from two or more distinct human donors. In some embodiments, each tissue within the array is substantially similar with regard to cell types, sources of cells, layers of cells, ratios of cells, methods of construction, size, shape, and the like. In other embodiments, one or more of the tissues within the array is unique with regard to cell types, sources of cells, layers of cells, ratios of cells, methods of construction, size, shape, and the like. In various embodiments, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, or more of the tissues within the array is unique. In other various embodiments, 1, 2, 3, 4, 5, 6, 7, 8, 9, 10, 15, 20, 25, 30, 35, 40, 45, 50, 55, 60, 65, 70, 75, 80, 85, 90, 91, 92, 93, 94, 95, 96, 97, 98, 99, or 100% of the tissues within the array is unique.

In some embodiments, one or more tissues within an array represent one or more specific tissues in the human body. In further embodiments, one or more individual tissues within an array represent human tissues including, by way of non-limiting example, blood or lymph vessel, muscle, uterus, nerve, mucous membrane, mesothelium, omentum, cornea, skin, liver, kidney, heart, trachea, lung, bone, bone marrow, adipose, connective tissue, bladder, breast, pancreas, spleen, brain, esophagus, stomach, intestine, colon, rectum, ovary, prostate, endoderm, ectoderm, and mesoderm. In one embodiment, the tissues within an array are selected to represent all the major tissue types in a subject.

In some embodiments, each tissue within the array is maintained independently in culture. In further embodiments, the culture conditions of each tissue within the array are such that they are isolated from the other tissues and cannot exchange media or factors soluble in the media. In other embodiments, two or more individual tissues within the array exchange soluble factors. In further embodiments, the culture conditions of two or more individual tissues within the array are such that they exchange media and factors soluble in the media with other tissues. In various embodiments, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, or more of the tissues within the array exchange media and/or soluble factors. In other various embodiments, 1, 2, 3, 4, 5, 6, 7, 8, 9, 10, 15, 20, 25, 30, 35, 40, 45, 50, 55, 60, 65, 70, 75, 80, 85, 90, 91, 92, 93, 94, 95, 96, 97, 98, 99, or 100% of the tissues within the array exchange media and/or soluble factors.

### In vitro Assays

In some embodiments, the engineered tissues, including connective tissue constructs, and arrays disclosed herein are for use in in vitro assays. In some embodiments, an “assay” is a procedure for testing or measuring the presence or activity of a substance (e.g., a chemical, molecule, biochemical, drug, etc.) in an organic or biologic sample (e.g., cell aggregate, tissue, organ, organism, etc.). In further embodiments, assays include qualitative assays and quantitative assays. In still further embodiments, a quantitative assay measures the amount of a substance in a sample.

In various embodiments, the engineered tissues, including connective tissue constructs, and arrays are for use in assays to detect or measure one or more of: molecular binding (including radioligand binding), molecular uptake, activity (e.g., enzymatic activity and receptor activity, etc.), gene expression, protein expression, receptor agonism, receptor antagonism, cell signaling, apoptosis, chemosensitivity, transfection, cell migration, chemotaxis, cell viability, cell proliferation, safety, efficacy, metabolism, toxicity, and abuse liability.

In some embodiments, the engineered tissues, including connective tissue constructs and arrays thereof are for use in immunoassays. In further embodiments, immunoassays are competitive immunoassays or noncompetitive immunoassays. In a competitive immunoassay, for example, the antigen in a sample competes with labeled antigen to bind with antibodies and the amount of labeled antigen bound to the antibody site is then measured. In a noncompetitive immunoassay (also referred to as a “sandwich assay”), for example, antigen in a sample is bound to an antibody site; subsequently, labeled antibody is bound to the antigen and the amount of labeled antibody on the site is then measured.

In some embodiments, the engineered tissues, including connective tissue constructs and arrays thereof are for use in enzyme-linked immunosorbent assays (ELISA). In further embodiments, an ELISA is a biochemical technique used to detect the presence of an antibody or an antigen in a sample. In ELISA, for example, at least one antibody with specificity for a particular antigen is utilized. By way of further example, a sample with an unknown amount of antigen is immobilized on a solid support (e.g., a polystyrene microtiter plate) either non-specifically (via adsorption to the surface) or specifically (via capture by another antibody specific to the same antigen, in a “sandwich” ELISA). By way of still further example, after the antigen is immobilized, the detection antibody is added, forming a complex with the antigen. The detection antibody can, for example, be covalently linked to an enzyme, or can itself be detected by a secondary antibody that is linked to an enzyme through bioconjugation.

For example, in some embodiments, an array, microarray, or chip of cells, multicellular aggregates, or tissues is used for drug screening or drug discovery. In further embodiments, an array, microarray, or chip of tissues is used as part of a kit for drug screening or drug discovery. In some embodiments, each connective tissue construct exists within a well of a biocompatible multi-well container, wherein the container is compatible with one or more automated drug screening procedures and/or devices. In further embodiments, automated drug screening procedures and/or devices include any suitable procedure or device that is computer or robot-assisted.

In further embodiments, arrays for drug screening assays or drug discovery assays are used to research or develop drugs potentially useful in any therapeutic area. In still further embodiments, suitable therapeutic areas include, by way of non-limiting examples, infectious disease, hematology, oncology, pediatrics, cardiology, central nervous system disease, neurology, gastroenterology, hepatology, urology, infertility, ophthalmology, nephrology, orthopedics, pain control, psychiatry, pulmonology, vaccines, wound healing, physiology, pharmacology, dermatology, gene therapy, toxicology, and immunology.

### Methods

Disclosed herein, in some embodiments, are methods of constructing tissues, including connective tissue constructs, comprising the steps of preparing bio-ink comprising connective tissue cells, optionally derived from mesenchymal stem/stromal cells; depositing the bio-ink onto a support; and incubating the bio-ink to allow the bio-ink to cohere and to form a living, three-dimensional connective tissue construct, wherein said incubation has a duration of about 1 hour to about 30 days. Also disclosed herein, in some embodiments, are methods of constructing tissues, including connective tissue constructs, comprising the steps of preparing bio-ink comprising mesenchymal stem/stromal cells; depositing the bio-ink onto a support; and incubating the bio-ink to allow the bio-ink to cohere and to form a living, three-dimensional connective tissue construct, wherein said incubation has a duration of about 1 hour to about 30 days. In some embodiments, the mesenchymal stem/stromal cells are exposed to one or more differentiation signals at one or more time intervals between about 1-21 days before depositing the bio-ink onto the support to about 1-21 days after depositing the bio-ink onto the support. In some embodiments, the methods utilize bioprinting. In further embodiments, the methods produce engineered tissues including connective tissue constructs, free or substantially free of any pre-formed scaffold at the time of use.

### Preparing Bio-Ink

In some embodiments, the methods involve preparing bio-ink comprising one or more types of mammalian cells. In further embodiments, the methods involve preparing bio-ink comprising connective tissue cells. In further embodiments, the methods involve preparing bio-ink comprising connective tissue cells, wherein the connective tissue cells are derived from mesenchymal stem/stromal cells. In some embodiments, the methods involve preparing bio-ink that further comprises mesenchymal stem/stromal cells. In further embodiments, the methods involve preparing bio-ink comprising mesenchymal stem/stromal cells, wherein the mesenchymal stem/stromal cells have been exposed to one or more differentiation signals. In some embodiments, the methods involve preparing bio-ink further comprising endothelial cells and/or fibroblasts.

There are various ways to make bio-ink having the characteristics described herein. In some embodiments, bio-ink is fabricated from a cell paste containing a plurality of living cells or with a desired cell density and viscosity. In further embodiments, the cell paste is shaped into a desired shape and a multicellular body formed through maturation (e.g., incubation). In a particular embodiment, an elongate multicellular body is produced by shaping a cell paste including a plurality of living cells into an elongate shape (e.g., a cylinder). In further embodiments, the cell paste is incubated in a controlled environment to allow the cells to adhere and/or cohere to one another to form the elongate multicellular body. In another particular embodiment, a multicellular body is produced by shaping a cell paste including a plurality of living cells in a device that holds the cell paste in a three-dimensional shape. In further embodiments, the cell paste is incubated in a controlled environment while it is held in the three dimensional shape for a sufficient time to produce a body that has sufficient cohesion to support itself on a flat surface.

In various embodiments, a cell paste is provided by: (A) mixing cells or cell aggregates (of one or more cell types) and a biocompatible gel or liquid, such as cell culture medium (e.g., in a pre-determined ratio) to result in a cell suspension, and (B) compacting the cellular suspension to produce a cell paste with a desired cell density and viscosity. In various embodiments, compacting is achieved by a number of methods, such as by concentrating a particular cell suspension that resulted from cell culture to achieve the desired cell concentration (density), viscosity, and consistency required for the cell paste. In a particular embodiment, a relatively dilute cell suspension from cell culture is centrifuged for a determined time to achieve a cell concentration in the pellet that allows shaping in a mold. Tangential flow filtration (“TFF”) is another suitable method of concentrating or compacting the cells. In some embodiments, compounds are combined with the cell suspension to lend the extrusion properties required. Suitable compounds include, by way of non-limiting examples, surfactant polyols, collagens, hydrogels, Matrigel™, nanofibers, self-assembling nanofibers, gelatin, fibrinogen, etc.

In some embodiments, the cell paste is produced by mixing a plurality of living cells with a tissue culture medium, and compacting the living cells (e.g., by centrifugation). One or more ECM components (or derivative of an ECM component) is optionally included by, resuspending the cell pellet in one or more physiologically acceptable buffers containing the ECM component(s) (or derivative(s) of ECM component(s)) and the resulting cell suspension centrifuged again to form a cell paste.

In some embodiments, the cell density of the cell paste desired for further processing may vary with cell types. In further embodiments, interactions between cells determine the properties of the cell paste, and different cell types will have a different relationship between cell density and cell-cell interaction. In still further embodiments, the cells may be pre-treated to increase cellular interactions before shaping the cell paste. For example, cells may be incubated inside a centrifuge tube after centrifugation in order to enhance cell-cell interactions prior to shaping the cell paste.

In various embodiments, many methods are used to shape the cell paste. For example, in a particular embodiment, the cell paste is manually molded or pressed (e.g., after concentration/compaction) to achieve a desired shape. By way of a further example, the cell paste is taken up (e.g., aspirated) into an instrument, such as a micropipette (e.g., a capillary pipette), that shapes the cell paste to conform to an interior surface of the instrument. The cross-sectional shape of the micropipette (e.g., capillary pipette) is alternatively circular, square, rectangular, triangular, or other non-circular cross-sectional shape. In some embodiments, the cell paste is shaped by depositing it into a preformed mold, such as a plastic mold, metal mold, or a gel mold. In some embodiments, centrifugal casting or continuous casting is used to shape the cell paste.

In some embodiments, substantially spherical multicellular aggregates, either alone or in combination with elongate cellular bodies, are also suitable to build the tissues, including connective tissue constructs, described herein. Spherical multicellular aggregates can be generated by a variety of methods, including, but not limited to, cellular self-assembly, the use of molds, and hanging drop methods. In further embodiments, a method to produce substantially spherical multicellular aggregates comprises the steps of 1) providing a cell paste containing a plurality of pre-selected cells or cell aggregates with a desired cell density and viscosity, 2) manipulating the cell paste into a cylindrical shape, 3) cutting cylinders into equal fragments, 4) letting the fragments round up overnight on a gyratory shaker, and 5) forming the substantially spherical multicellular aggregates through maturation.

In some embodiments, a partially adhered and/or cohered cell paste is transferred from the shaping device (e.g., capillary pipette) to a second shaping device (e.g., a mold) that allows nutrients and/or oxygen to be supplied to the cells while they are retained in the second shaping device for an additional maturation period. One example of a suitable shaping device that allows the cells to be supplied with nutrients and oxygen is a mold for producing a plurality of multicellular aggregates (e.g., substantially identical multicellular aggregates). By way of further example, such a mold includes a biocompatible substrate made of a material that is resistant to migration and ingrowth of cells into the substrate and resistant to adherence of cells to the substrate. In various embodiments, the substrate can suitably be made of Teflon®, (PTFE), stainless steel, agarose, polyethylene glycol, glass, metal, plastic, or gel materials (e.g., agarose gel or other hydrogel), and similar materials. In some embodiments, the mold is also suitably configured to allow supplying tissue culture media to the cell paste (e.g., by dispensing tissue culture media onto the top of the mold).

Thus, in embodiments where a second shaping device is used, the partially adhered and/or cohered cell paste is transferred from the first shaping device (e.g., a capillary pipette) to the second shaping device (e.g., a mold). In further embodiments, the partially adhered and/or cohered cell paste can be transferred by the first shaping device (e.g., the capillary pipette) into the grooves of a mold. In still further embodiments, following a maturation period in which the mold is incubated along with the cell paste retained therein in a controlled environment to allow the cells in the cell paste to further adhere and/or cohere to one another to form the multicellular aggregate, the cohesion of the cells will be sufficiently strong to allow the resulting multicellular aggregate to be picked up with an implement (e.g., a capillary pipette). In still further embodiments, the capillary pipette is suitably be part of a printing head of a bioprinter or similar apparatus operable to automatically place the multicellular aggregate into a three-dimensional construct.

In some embodiments, the cross-sectional shape and size of the multicellular aggregates will substantially correspond to the cross-sectional shapes and sizes of the first shaping device and optionally the second shaping device used to make the multicellular aggregates, and the skilled artisan will be able to select suitable shaping devices having suitable cross-sectional shapes, cross-sectional areas, diameters, and lengths suitable for creating multicellular aggregates having the cross-sectional shapes, cross-sectional areas, diameters, and lengths discussed above.

In some embodiments, the method of bioprinting is continuous and/or substantially continuous. A non-limiting example of a continuous bioprinting method is to dispense bio-ink from a bioprinter via a dispense tip (e.g., a needle, capillary tube, etc.) connected to a reservoir of bio-ink. In some embodiments, the cell paste is loaded into a reservoir and bioprinted directly into a receptacle or support with a defined shape. In further embodiments, the receptacle or support enables formation of bio-ink within about 15 minutes to about 6 hours after deposition. In further embodiments, the receptacle or support is suitable for both the formation of bio-ink and the formation of a three-dimensional tissue. In further embodiments, the receptacle or support is compatible with in vitro maintenance and maturation of the three-dimensional tissue after fabrication. In some embodiments, one or more cell pastes are bioprinted in a defined pattern directly into a receptacle or support. In some embodiments, multiple bio-inks are deposited in a specific pattern, thereby generating a specific planar geometry in the x- and y-axes in each layer of tissue. In still further embodiments, a first bioink is utilized to create a geometric or user-defined pattern via a dispensed series of lines or borders, and additional distinct bio-inks are utilized as fills within the borders created by the first bioink. In still further embodiments, borders can be created by two or more distinct bio-inks, and two or more distinct bio-inks are utilized as fills within the borders of the pattern. The resulting tissue is a mosaic, or compartmentalized tissue that resembles a stained glass window, consisting of borders (e.g., frames) and fills (e.g., panes). In further embodiments, multiple layers can be added atop the first layer, with each layer comprising the same geometry of the first layer or a distinct geometry from the first layer.

### Depositing Bio-Ink onto a Support

A number of methods are suitable to deposit bio-ink onto a support to produce a desired three-dimensional structure. For example, in some embodiments, the multicellular aggregates are manually placed in contact with one another, deposited in place by extrusion from a pipette, nozzle, or needle, or positioned by an automated, computer-assisted device such as a bioprinter.

As described herein, in various embodiments, bio-ink comprises multicellular aggregates having many suitable shapes and sizes. In some embodiments, multicellular aggregates are elongate with any of several suitable cross-sectional shapes including, by way of non-limiting example, circular, oval, square, triangular, polygonal, and irregular. In further embodiments, multicellular aggregates are elongate and in the form of a cylinder. In some embodiments, elongate multicellular aggregates are of similar lengths and/or diameters. In other embodiments, elongate multicellular aggregates are of differing lengths and/or diameters. In some embodiments, multicellular aggregates are substantially spherical. In some embodiments, the engineered tissues (e.g., connective tissue constructs, etc.) include substantially spherical multicellular aggregates that are substantially similar in size. In other embodiments, the engineered tissues (e.g., connective tissue constructs, etc.) include substantially spherical multicellular aggregates that are of differing sizes. In some embodiments, engineered tissues (e.g., connective tissue constructs, etc.) of different shapes and sizes are formed by arranging multicellular aggregates of various shapes and sizes.

In some embodiments, the cohered multicellular aggregates are deposited onto a support. In various embodiments, the support is any suitable biocompatible surface. In still further embodiments, suitable biocompatible surfaces include, by way of non-limiting examples, polymeric material, porous membranes, plastic, glass, metal, hydrogel, and combinations thereof. In some embodiments, the support is coated with a biocompatible substance including, by way of non-limiting examples, a hydrogel, a protein, a chemical, a peptide, antibodies, growth factors, or combinations thereof. In one embodiment, the support is coated with NovoGel™. In another embodiment, the support is coated with agarose. In one embodiment, the cohered multicellular aggregates are placed into the wells of a biocompatible multi-well container.

Once deposition of the bio-ink is complete, in some embodiments, a tissue culture medium is poured over the top of the construct. In further embodiments, the tissue culture medium enters the spaces between the multicellular bodies to support the cells in the multicellular bodies.

### Incubating Bio-Ink and/or Tissue Constructs

In some embodiments, the deposited bio-ink is incubated. In further embodiments, incubation allows the bio-ink to cohere and form a living, three-dimensional connective tissue construct. In some embodiments, the bio-ink coheres to form a tissue in a cell culture environment (e.g., a Petri dish, cell culture flask, bioreactor, etc.). In further embodiments, the bio-ink coheres to form a tissue in an environment with conditions suitable to facilitate growth of the cell types included in the bio-ink. In one embodiment, the bio-ink/tissue construct is incubated at about 37° C., in a humidified atmosphere containing about 5% CO₂, in the presence of cell culture medium containing factors and/or ions to foster adherence and/or coherence. In other embodiments, the bio-ink/tissue construct is maintained in an environment that contains 0.1%-21% O₂.

The incubation, in various embodiments, has any suitable duration. In further various embodiments, the incubation has a duration of about 20, 30, 40, 50, 60, 70, 80, 90, 100, 110, 120, 130, 140, 150, 160, 170, 180, or more minutes, including increments therein. In further various embodiments, the incubation has a duration of about 1, 2, 3, 4, 5, 6, 7, 8, 9, 10, 11, 12, 13, 14, 15, 16, 17, 18, 19, 20, 21, 22, 23, 24, 36, 48, or more hours, including increments therein. In further various embodiments, the incubation has a duration of about 1, 2, 3, 4, 5, 6, 7, 8, 9, 10, 11, 12, 13, 14, 15, 16, 17, 18, 19, 20, or more days, including increments therein. Several factors influence the time required for bio-ink to cohere to form a tissue including, by way of non-limiting examples, cell types, cell type ratios, culture conditions, and the presence of additives such as growth factors.

### Additional Steps for Increasing Viability of Engineered Tissue

In some embodiments, the method further comprises steps for increasing the viability of the engineered tissue. In further embodiments, these steps involve providing direct contact between the tissue and a nutrient medium through a temporary or semi-permanent lattice of confinement material. In some embodiments, the tissue is constrained in a porous or gapped material. Direct access of at least some of the cells of the engineered tissue to nutrients increases the viability of the engineered tissue.

In further embodiments, the additional and optional steps for increasing the viability of the engineered tissue include: 1) optionally dispensing a base layer of confinement material prior to placing cohered multicellular aggregates; 2) optionally dispensing a perimeter of confinement material; 3) bioprinting cells of the tissue within a defined geometry; and 4) dispensing elongate bodies (e.g., cylinders, ribbons, etc.) of confinement material overlaying the nascent tissue in a pattern that introduces gaps in the confinement material, such as a lattice, mesh, or grid.

Many confinement materials are suitable for use in the methods described herein. In some embodiments, hydrogels are exemplary confinement materials possessing one or more advantageous properties including: non-adherent, biocompatible, extrudable, bioprintable, non-cellular, of suitable strength, and not soluble in aqueous conditions. In some embodiments, suitable hydrogels are natural polymers. In one embodiment, the confinement material is comprised of NovoGel™. In further embodiments, suitable hydrogels include those derived from surfactant polyols such as Pluronic F-127, collagen, hyaluronate, fibrin, gelatin, peptide hydrogels, alginate, agarose, chitosan, and derivatives or combinations thereof. In other embodiments, suitable hydrogels are synthetic polymers. In further embodiments, suitable hydrogels include those derived from poly(acrylic acid) and derivatives thereof, poly(ethylene oxide) and copolymers thereof, poly(vinyl alcohol), polyphosphazene, and combinations thereof. In various specific embodiments, the confinement material is selected from: hydrogel, NovoGel™, agarose, alginate, gelatin, Matrigel™, hyaluronan, poloxamer, peptide hydrogel, poly(isopropyl n-polyacrylamide), polyethylene glycol diacrylate (PEG-DA), hydroxyethyl methacrylate, polydimethylsiloxane, polyacrylamide, poly(lactic acid), silicon, silk, or combinations thereof.

In some embodiments, the gaps overlaying pattern are distributed uniformly or substantially uniformly around the surface of the tissue. In other embodiments, the gaps are distributed non-uniformly, whereby the cells of the tissue are exposed to nutrients non-uniformly. In non-uniform embodiments, the differential access to nutrients may be exploited to influence one or more properties of the tissue. For instance, it may be desirable to have cells on one surface of a bioprinted tissue proliferate faster than cells on another surface of the bioprinted tissue. In some embodiments, the exposure of various parts of the tissue to nutrients can be changed at various times to influence the development of the tissue toward a desired endpoint.

In some embodiments, the confinement material is removed at any suitable time, including but not limited to, immediately after bioprinting (e.g., within 10 minutes), after bioprinting (e.g., after 10 minutes), before the cells are substantially cohered to each other, after the cells are substantially cohered to each other, before the cells produce an extracellular matrix, after the cells produce an extracellular matrix, just prior to use, and the like. In various embodiments, confinement material is removed by any suitable method. For example, in some embodiments, the confinement material is excised, pulled off the cells, digested, or dissolved.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

### Various Non-Limiting Embodiments

In some embodiments, disclosed herein are living, three-dimensional connective tissue constructs comprising: connective tissue cells cohered to one another to provide a living, three-dimensional connective tissue construct; wherein the construct is substantially free of pre-formed scaffold at the time of use. In some embodiments, the connective tissue cells are derived from mesenchymal stem/stromal cells. In further embodiments, the mesenchymal stem/stromal cells are derived from mammalian adipose tissue. In further embodiments, the mesenchymal stem/stromal cells are derived from mammalian bone marrow. In other embodiments, the mesenchymal stem/stromal cells are derived from a non-adipose, non-bone marrow tissue source. In some embodiments, the mesenchymal stem/stromal cells were exposed to the one or more differentiation signals before fabrication of the construct. In some embodiments, the mesenchymal stem/stromal cells were exposed to the one or more differentiation signals during fabrication of the construct. In some embodiments, the mesenchymal stem/stromal cells were exposed to the one or more differentiation signals after fabrication of the construct. In some embodiments, the construct was bioprinted. In further embodiments, the construct further comprises an extrusion compound, the extrusion compound improving the suitability of the cells for bioprinting. In some embodiments, the connective tissue is selected from the group consisting of: bone, cartilage, tendon, and ligament. In some embodiments, the construct further comprises mammalian endothelial cells. In further embodiments, the ratio of connective tissue cells to endothelial cells is between about 5:1 to about 20:1. In still further embodiments, the ratio of connective tissue cells to endothelial cells is about 9:1. In some embodiments, the construct further comprises mammalian fibroblasts. In some embodiments, the construct is in the form of a sheet or patch. In some embodiments, the construct further comprises one or more of discrete filler bodies, each filler body comprising a biocompatible material, wherein the one or more filler body creates a gap or space in the cohered cells. In particular embodiments, each filler body substantially resists migration and ingrowth of cells.

In some embodiments, disclosed herein are living, three-dimensional connective tissue constructs comprising: mesenchymal stem/stromal cells cohered to one another, wherein the mesenchymal stem/stromal cells have been exposed to one or more differentiation signals to provide a living, three-dimensional connective tissue construct; wherein the construct is substantially free of pre-formed scaffold. In some embodiments, a construct was bioprinted. In some embodiments, a construct further comprises an extrusion compound, the extrusion compound improving the suitability of the cells for bioprinting. In some embodiments, the connective tissue is selected from the group consisting of: bone, cartilage, tendon, and ligament. In further embodiments, the connective tissue is bone. In some embodiments, a construct further comprises mammalian endothelial cells. In further embodiments, the ratio of mesenchymal stem/stromal cells to endothelial cells is between about 5:1 to about 20:1. In still further embodiments, the ratio of mesenchymal stem/stromal cells to endothelial cells is about 9:1. In some embodiments, a construct further comprises mammalian fibroblasts. In some embodiments, the mesenchymal stem/stromal cells are derived from mammalian adipose tissue. In some embodiments, the mesenchymal stem/stromal cells are derived from mammalian bone marrow. In other embodiments, the mesenchymal stem/stromal cells are derived from a non-adipose, non-bone marrow tissue source. In some embodiments, the cells were exposed to the one or more differentiation signals before fabrication of the construct. In some embodiments, the cells were exposed to the one or more differentiation signals during fabrication of the construct. In some embodiments, the cells were exposed to the one or more differentiation signals after fabrication of the construct. In some embodiments, a construct is in the form of a sheet or patch. In some embodiments, a construct further comprises one or more of discrete filler bodies, each filler body comprising a biocompatible material that substantially resists migration and ingrowth of cells, wherein the one or more filler body creates a gap or space in the cohered cells. In some embodiments, the one or more differentiation signals comprise incubation in differentiation media. In some embodiments, the one or more differentiation signals comprise mechanical, biomechanical, or physical signals, or combinations thereof. In some embodiments, some portion of the mesenchymal stem/stromal cells are characterized by partial or complete differentiation toward a cell type present in mammalian connective tissue.

In some embodiments, disclosed herein are living, three-dimensional connective tissue constructs comprising: mesenchymal stem/stromal cells, fibroblasts, and endothelial cells, wherein the cells are cohered to one another, wherein the mesenchymal stem/stromal cells have been exposed to one or more differentiation medias at one or more time intervals between about 1-21 days before fabrication of the construct to about 1-21 days after fabrication of the construct to provide a living, three-dimensional connective tissue construct; wherein the connective tissue construct is substantially free of pre-formed scaffold.

In some embodiments, disclosed herein are living, three-dimensional connective tissue constructs, the constructs comprising mammalian cells, the construct fabricated by a process comprising: exposing mesenchymal stem/stromal cells to one or more differentiation signals to provide a living, three-dimensional connective tissue construct, wherein the construct consists essentially of cellular material and is implantable in a subject. In some embodiments, the cells were bioprinted. In some embodiments, a construct is substantially free of any pre-formed scaffold. In some embodiments, the connective tissue is selected from the group consisting of: bone, cartilage, tendon, and ligament. In further embodiments, the connective tissue is bone. In some embodiments, a construct is for implantation in the subject at a site of injury, disease, or degeneration. In some embodiments, a construct further comprises mammalian endothelial cells. In further embodiments, the ratio of mesenchymal stem/stromal cells to endothelial cells is between about 5:1 to about 20:1. In still further embodiments, the ratio of mesenchymal stem/stromal cells to endothelial cells is about 9:1. In some embodiments, a construct further comprises mammalian fibroblasts. In some embodiments, the construct is a compound tissue construct comprising one or more connective tissues. In further embodiments, the construct is a compound tissue construct comprising connective tissue and a non-connective tissue. In still further embodiments, the construct is a compound tissue construct comprising bone tissue and a non-connective tissue. In some embodiments, the one or more differentiation signals comprise incubation in differentiation media. In some embodiments, the one or more differentiation signals comprise mechanical, biomechanical, or physical signals, or combinations thereof.

In some embodiments, disclosed herein are arrays of living, three-dimensional connective tissue constructs, each construct comprising mammalian cells, each construct fabricated by a process comprising: exposing mesenchymal stem/stromal cells to one or more differentiation signals to provide a living, three-dimensional connective tissue construct; wherein each connective tissue construct is substantially free of pre-formed scaffold at the time of use; wherein each connective tissue construct is maintained in culture. In some embodiments, each construct in an array was bioprinted. In some embodiments, the connective tissue is selected from the group consisting of: bone, cartilage, tendon, and ligament. In further embodiments, the connective tissue is bone. In some embodiments, one or more connective tissue constructs in an array further comprises mammalian endothelial cells. In further embodiments, the ratio of mesenchymal stem/stromal cells to endothelial cells is between about 5:1 to about 20:1. In still further embodiments, the ratio of mesenchymal stem/stromal cells to endothelial cells is about 9:1. In some embodiments, one or more connective tissue constructs in an array further comprises mammalian fibroblasts. In some embodiments, one or more connective tissue constructs in an array are compound tissue constructs comprising one or more connective tissues. In some embodiments, one or more connective tissue constructs in an array are compound tissue constructs comprising connective tissue and a non-connective tissue. In further embodiments, one or more connective tissue constructs in an array are compound tissue constructs comprising bone tissue and a non-connective tissue. In some embodiments, an array is for use in in vitro assays. In further embodiments, an array is for use in one or more selected from the group consisting of: drug discovery, drug testing, toxicology testing, disease modeling, three-dimensional biology studies, and cell screening. In some embodiments, the one or more differentiation signals comprise incubation in differentiation media. In some embodiments, the one or more differentiation signals comprise mechanical, biomechanical, or physical signals, or combinations thereof.

In some embodiments, disclosed herein are methods of fabricating living, three-dimensional connective tissue constructs comprising the steps of: preparing bio-ink comprising mesenchymal stem/stromal cells; depositing the bio-ink onto a support; and incubating the bio-ink to allow the bio-ink to cohere and to form a living, three-dimensional connective tissue construct, wherein said incubation has a duration of about 1 hour to about 30 days; with the proviso that the mesenchymal stem/stromal cells are exposed to one or more differentiation signals at one or more time intervals between about 1-21 days before depositing the bio-ink onto the support to about 1-21 days after depositing the bio-ink onto the support. In some embodiments, the bio-ink is deposited by bioprinting. In some embodiments, the construct is substantially free of any pre-formed scaffold. In some embodiments, the connective tissue is selected from the group consisting of: bone, cartilage, tendon, and ligament. In further embodiments, the connective tissue is bone. In some embodiments, the bio-ink further comprises mammalian endothelial cells. In further embodiments, the ratio of mesenchymal stem/stromal cells to endothelial cells is between about 5:1 to about 20:1. In still further embodiments, the ratio of mesenchymal stem/stromal cells to endothelial cells is about 9:1. In some embodiments, the bio-ink further comprises mammalian fibroblasts. In some embodiments, the bio-ink further comprises an extrusion compound. In some embodiments, the mesenchymal stem/stromal cells are derived from mammalian adipose tissue. In some embodiments, the mesenchymal stem/stromal cells are derived from mammalian bone marrow. In other embodiments, the mesenchymal stem/stromal cells are derived from a non-adipose, non-bone marrow tissue source. In some embodiments, the one or more differentiation signals comprise incubation in a differentiation media. In some embodiments, the one or more differentiation signals comprise mechanical, biomechanical, or physical signals, or combinations thereof. In some embodiments, the method further comprises the step of depositing one or more discrete filler bodies, each filler body comprising a biocompatible material that substantially resists migration and ingrowth of cells, wherein the one or more filler body creates a gap or space in the cohered cells. In some embodiments, the method further comprises the step of assembling a plurality of living, three-dimensional connective tissue constructs into an array by attaching the constructs to a biocompatible surface. In further embodiments, the biocompatible surface is a porous membrane.

### EXAMPLES

The following specific examples are to be construed as merely illustrative, and not limitative of the remainder of the disclosure in any way whatsoever. Without further elaboration, it is believed that one skilled in the art can, based on the description herein, utilize the present invention to its fullest extent.

### Example 1

### MSC Culture

MSCs were cultured and expanded in standard cell culture conditions using a basal media that contained 5-10% (v:v) fetal bovine serum in low glucose DMEM supplemented with L-glutamine. In some cases, the MSCs were cultured in low (3-5%) oxygen conditions.

### Example 2

### NovoGel™ Solutions and Mold

### Preparation of 2% and 4% (w/v) NovoGel™ Solution

1 g or 2 g (for 2% or 4% respectively) of NovoGel™ (Organovo, San Diego, Calif.) was dissolved in 50 ml of Dulbecco's phosphate buffered saline (DPBS; Invitrogen Corp., Carlsbad, Calif.). Briefly, the DPBS and NovoGel™ are heated to 85° C. on a hot plate with constant stirring until the NovoGel™ dissolves completely. NovoGel™ solution is sterilized by steam sterilization at 125° C. for 25 minutes. The NovoGel™ solution remains in liquid phase as long as the temperature is maintained above 36.5° C. Below this temperature a phase transition occurs, the viscosity of the NovoGel™ solution increases and the NovoGel™ forms a solid gel.

### Preparation of NovoGel™ Mold

A NovoGel™ mold was fabricated for the incubation of bio-ink (in the form of cellular cylinders) using a Teflon® mold that fit a 10 cm Petri dish. Briefly, the Teflon® mold was pre-sterilized using 70% ethanol solution and subjecting the mold to UV light for 45 minutes. The sterilized mold was placed on top of the 10 cm Petri dish (VWR International LLC, West Chester, Pa.) and securely attached. This assembly (Teflon® mold+Petri dish) was maintained vertically and 45 ml of pre-warmed, sterile 2% NovoGel™ solution was poured in the space between the Teflon® mold and the Petri dish. The assembly was then placed horizontally at room temperature for 1 hour to allow complete gelation of the NovoGel™. After gelation, the Teflon® print was removed and the NovoGel™ mold was washed twice using DPBS. Then 17.5 ml of HASMC culture medium was added to the NovoGel™ mold for incubating the bio-ink.

### Example 3

### Fabrication of MSC-HAEC Bio-Ink

To prepare bio-ink (in the form of mixed cellular cylinders) MSC and HAEC were individually collected and then mixed at pre-determined ratios. Briefly, the culture medium was removed from confluent culture flasks and the cells were washed with DPBS (1 ml/5 cm² of growth area). Cells were detached from the surface of the culture flasks by incubation in the presence of trypsin (1 ml/15 cm² of growth area; Invitrogen Corp., Carlsbad, Calif.) for 10 minutes. MSC were detached using 0.15% trypsin while HAEC were detached using 0.1% trypsin. Following the incubation appropriate culture medium was added to the flasks (2× volume with respect to trypsin volume). The cell suspension was centrifuged at 200 g for 6 minutes followed by complete removal of supernatant solution. Cell pellets were resuspended in respective culture medium and counted using a hemocytometer. Appropriate volumes of MSC and HAEC were combined to yield a mixed cell suspension containing 10% HAEC and remainder 90% MSC (as a % of total cell population). The mixed cell suspension was centrifuged at 200 g for 5 minutes followed by complete removal of supernatant solution. Mixed cell pellets were resuspended in 6 ml of MSC culture medium and transferred to 20 ml glass vials (VWR International LLC, West Chester, Pa.), followed by incubation on an orbital shaker at 150 rpm for 60 minutes, and at 37° C. and 5% CO2. This allows the cells to aggregate with one another and initiate cell-cell adhesions. Post-incubation, the cell suspension was transferred to a 15 ml centrifuge tube and centrifuged at 200 g for 5 minutes. After removal of the supernatant medium, the cell pellet was resuspended in 400 μl of MSC culture medium and pipetted up and down several times to ensure all cell clusters were broken. The cell suspension was transferred into a 0.5 ml microfuge tube (VWR International LLC, West Chester, Pa.) placed inside a 15 ml centrifuge tube followed by centrifugation at 2000 g for 4 minutes to form a highly dense and compact cell pellet. The supernatant medium was aspirated and the cells were transferred into capillary tubes (OD 1.5 mm, ID 0.5 mm, L 75 mm; Drummond Scientific Co., Broomall, Pa.) by aspiration so as to yield cellular cylinders 50 mm in length. The cell paste inside the capillaries was incubated in MSC medium for 20 minutes at 37° C. and 5% CO₂. The cellular cylinders were then extruded from the capillary tubes into the grooves of a NovoGel™ mold (covered with MSC medium) using the plunger supplied with the capillaries. The bio-ink was incubated for 24 hours at 37° C. and 5% CO₂.

### Example 4

### Pre-Deposition MSC Differentiation Via Incubation with Osteogenic Differentiation Media

Cultured MSC were treated with 1× osteogenic differentiation media (CombiCult™ Media; Plasticell, Inc., London, UK) continuously for 5 days prior to deposition with a bioprinter. On day −1 the MSC were used to produce bio-ink using methods described herein. On day 0, the bio-ink was bioprinted to form a tissue construct. Subsequent to bio-ink fusion the construct was assessed for cell differentiation.

### Example 5

### Bioprinting of Connective Tissue Construct

Engineered connective tissue constructs were bioprinted utilizing a NovoGen MMX Bioprinter™ (Organovo, Inc., San Diego, Calif.) using a bio-ink extrusion mechanism. The bio-ink was composed of MSCs and human aortic endothelial cells (HAECs) in a ratio of 90% MSC:10% HAEC. The construct was bioprinted directly onto a Transwell® permeable support membrane in the form of a 5 mm×8 mm sheet.

### Example 6

### Peri-Deposition MSC Differentiation Via Incubation with Osteogenic Differentiation Media

Cultured MSC were treated with osteogenic differentiation media according to the following experimental protocol:

1) No pre-deposition exposure; exposure to 1× osteogenic differentiation media beginning on day 0 (deposition) and continuing until 3 days or 6 days post-deposition.

2) Pre-deposition exposure to 0.5× osteogenic differentiation media beginning 3 days prior to deposition and continuing until 3 days or 6 days post-deposition.

3) Pre-deposition exposure to 1× osteogenic differentiation media beginning 3 days prior to deposition and continuing until 3 days or 6 days post-deposition.

Subsequent to bio-ink fusion the construct was assessed for cell differentiation.

### Example 7

### Assessment of MSC Differentiation

Connective tissue constructs comprising MSC exposed to osteogenic differentiation media were assessed for degree of connective tissue-specific differentiation. The constructs were sectioned, fixed in formalin, embedded in paraffin, and subjected to Alizarin Red S staining (stains calcium crystals) and Von Kossa staining (stains calcium phosphate deposits) followed by microscopy. The constructs were also subjected to alkaline phosphatase staining and ELISA for osteopontin expression. See, e.g., FIGS. 2 and 3.

What is claimed is:

1. An engineered, living, three-dimensional connective tissue construct comprising: connective tissue cells cohered to one another to provide a living, three-dimensional connective tissue construct; wherein the construct is substantially free of pre-formed scaffold at the time of use.

2. The construct of claim 1, wherein the construct is non-innervated.

3. The construct of claim 1, wherein the connective tissue cells comprise connective tissue cells derived in vitro from multi-potent cells.

4. The construct of claim 3, wherein the multi-potent cells comprise one or more of: tissue-specific progenitors, mesenchymal stem/stromal cells, induced pluripotent stem cells, and embryonic stem cells.

5. The construct of claim 3, wherein the multi-potent cells are derived from mammalian adipose tissue.

6. The construct of claim 3, wherein the multi-potent cells are derived from mammalian bone marrow.

7. The construct of claim 3, wherein the multi-potent cells are derived from a non-adipose, non-bone marrow tissue source.

8. The construct of claim 3, wherein the multi-potent cells were exposed to one or more differentiation signals before fabrication of the construct.

9. The construct of claim 3, wherein the multi-potent cells were exposed to one or more differentiation signals during fabrication of the construct.

10. The construct of claim 3, wherein the multi-potent cells were exposed to one or more differentiation signals after fabrication of the construct.

11. The construct of claim 1, wherein the construct was bioprinted.

12. The construct of claim 11, further comprising an extrusion compound, the extrusion compound improving the suitability of the cells for bioprinting.

13. The construct of claim 1, wherein the connective tissue is selected from the group consisting of: bone, cartilage, tendon, and ligament.

14. The construct of claim 1, further comprising one or more of the following cell types:
vascular, endothelial, fibroblasts, pericytes, stem/progenitor cells, immune cells.

15. The construct of claim 1, substantially in the form of a sheet, patch, ring, tube, cube, polyhedron, or sphere.

16. The construct of claim 1, substantially in the form of a shape that mimics the shape or architecture of a native human connective tissue in vivo.

17. The construct of claim 1, for implantation in a subject at a site of injury, disease, or degeneration.

18. The construct of claim 1, further comprising one or more of discrete filler bodies, each filler body comprising a biocompatible material, wherein the one or more filler body creates a gap or space in the cohered cells.

19. The construct of claim 18, wherein each filler body substantially resists migration and ingrowth of cells.

20. An array of engineered, living, three-dimensional connective tissue constructs, each construct fabricated by a process comprising: exposing multi-potent cells to one or more differentiation signals to provide a living, three-dimensional connective tissue construct; wherein each connective tissue construct is substantially free of pre-formed scaffold at the time of use;
wherein each connective tissue construct is maintained in culture.

21. The array of claim 20, wherein each construct is non-innervated.

22. The array of claim 20, wherein the multi-potent cells comprise one or more of: tissue-specific progenitors, mesenchymal stem/stromal cells, induced pluripotent stem cells, and embryonic stem cells.

23. The array of claim 20, wherein the multi-potent cells are derived from mammalian adipose tissue.

24. The array of claim 20, wherein the multi-potent cells are derived from mammalian bone marrow.

25. The array of claim 20, wherein the multi-potent cells are derived from a non-adipose, non-bone marrow tissue source.

26. The array of claim 20, wherein the multi-potent cells were exposed to the one or more differentiation signals before fabrication of the construct.

27. The array of claim 20, wherein the multi-potent cells were exposed to the one or more differentiation signals during fabrication of the construct.

28. The array of claim 20, wherein the multi-potent cells were exposed to the one or more differentiation signals after fabrication of the construct.

29. The array of claim 20, wherein each construct was bioprinted.

30. The array of claim 20, wherein the connective tissue is selected from the group consisting of: bone, cartilage, tendon, and ligament.

31. The array of claim 20, wherein one or more connective tissue constructs further comprises one or more of the following cell types: endothelial cells, fibroblasts, stem/progenitor cells, pericytes, satellite cells, or vascular cells.

32. The array of claim 20, wherein one or more connective tissue constructs are compound tissue constructs comprising one or more connective tissues.

33. The array of claim 32, wherein one or more connective tissue constructs are compound tissue constructs comprising connective tissue and a non-connective tissue.

34. The array of claim 33, wherein one or more connective tissue constructs are compound tissue constructs comprising bone tissue and a non-connective tissue.

35. The array of claim 20, for use in in vitro assays.

36. The array of claim 35, for use in one or more of: drug discovery, drug testing, toxicology testing, disease modeling, three-dimensional biology studies, and cell screening.

37. The array of claim 20, wherein the one or more differentiation signals comprise mechanical, biomechanical, soluble, or physical signals, or combinations thereof.

38. The array of claim 20, wherein one or more constructs further comprises one or more discrete filler bodies, each filler body comprising a biocompatible material, wherein the one or more filler body creates a gap or space in the cohered cells.

39. The array of claim 38, wherein each filler body substantially resists migration and ingrowth of cells.

40. A method of fabricating a living, three-dimensional connective tissue construct comprising:
incubating a bio-ink, comprising multi-potent cells that have been deposited on a support and exposed to one or more differentiation signals, to allow the bio-ink to cohere and to form a living, three-dimensional connective tissue construct, wherein said incubation has a duration of about 1 hour to about 30 days.

41. The method of claim 40, wherein the multi-potent cells comprise one or more of:
mesenchymal stem/stromal cells, induced pluripotent stem cells, and embryonic stem cells.

42. The method of claim 40, wherein the multi-potent cells are derived from mammalian adipose tissue.

43. The method of claim 40, wherein the multi-potent cells are derived from mammalian bone marrow.

44. The method of claim 40, wherein the multi-potent cells are derived from a non-adipose, non-bone marrow tissue source.

45. The method of claim 40, wherein the connective tissue cells are exposed to one or more differentiation signals at one or more time intervals between about 1-21 days before depositing the bio-ink onto the support to about 1-21 days after depositing the bio-ink onto the support.

46. The method of claim 40, wherein the bio-ink is deposited by bioprinting.

47. The method of claim 40, wherein the construct is substantially free of any pre-formed scaffold at the time of use.

48. The method of claim 40, wherein the construct is non-innervated.

49. The method of claim 40, wherein the connective tissue is selected from the group consisting of: bone, cartilage, tendon, and ligament.

50. The method of claim 40, wherein the bio-ink further comprises one or more of the following cell types: vascular, endothelial, fibroblasts, pericytes, stem/progenitor cells, immune cells.

51. The method of claim 40, wherein the bio-ink further comprises an extrusion compound.

52. The method of claim 40, wherein the one or more differentiation signals comprise mechanical, biomechanical, soluble, or physical signals, or combinations thereof.

53. The method of claim 40, further comprising depositing one or more discrete filler bodies, each filler body comprising a biocompatible material, wherein the one or more filler body creates a gap or space in the cohered cells.

54. The method of claim 53, wherein each filler body substantially resists migration and ingrowth of cells.

55. The method of claim 40, further comprising assembling a plurality of living, three-dimensional connective tissue constructs into an array by spatially confining the constructs onto or within a biocompatible surface.

56. The method of claim 40, wherein the construct is suitable for implantation in a subject at a site of injury, disease, or degeneration.
